(19) United States
(12) Patent Application Publication
(10) Pub. No.: US 2026/0273067 A1
(43) Pub. Date: Sep. 17, 2026

(54) GLUCOSE SENSITIVE INSULIN DERIVATIVES

(71) Applicant: Novo Nordisk A/S, Bagsvaerd (DK)

(72) Inventors: Thomas Hoeg-Jensen, Pasadena, CA (US); Line Due Buron, Soeborg (DK); Thomas Boerglum Kjeldsen, Oesterbro (DK); Henrik Valore, Espergaerde (DK); Alice Ravn Madsen, Roedovre (DK); Per Sauerberg, Farum (DK)

(21) Appl. No.: 19/474,481

(22) PCT Filed: Apr. 23, 2024
(86) PCT No.: PCT/EP2024/060997

§ 371 Date: Oct. 10, 2025

(30) Foreign Application Priority Data
Apr. 24, 2023 (EP) 23169455.5
Sep. 27, 2023 (EP) 23199974.9

(51) Int. Cl. A61K 47/54; A61K 38/28; A61P 3/10

(52) U.S. Cl. CPC A61K 47/545; A61K 38/28; A61P 3/10

(57) ABSTRACT

The present invention relates to novel insulin derivatives and their use in the treatment or prevention of medical conditions relating to diabetes. The insulin derivatives are glucose sensitive and display glucose-sensitive albumin binding. The insulin derivatives have a long half-life. The invention also relates to novel intermediates. Finally, the invention provides a pharmaceutical composition comprising the insulin derivatives of the invention and the use of such a composition in the treatment or prevention of medical conditions relating to diabetes.

### TECHNICAL FIELD

The present invention relates to novel insulin derivatives, and their pharmaceutical use. Furthermore, the invention relates to pharmaceutical compositions comprising such insulin derivatives, and to the use of such compounds for the treatment or prevention of medical conditions relating to diabetes.

### INCORPORATION-BY-REFERENCE OF THE SEQUENCE LISTING

### Sequence Listing

The present application is filed with a Sequence Listing in electronic form. The entire contents of the sequence listing are hereby incorporated by reference.

### BACKGROUND

Insulin is the most effective drug for treatment of high blood glucose, but insulin dosing is a delicate balance between too much and too little since the physiological glucose window is narrow. Healthy persons have glucose levels at fasted state near 5 mM, and people living with diabetes try to dose both meal and basal insulin preparations to get near 5 mM. However, blood glucose values below approximately 3 mM (hypoglycaemia) often occur during insulin treatments, and hypoglycaemia can result in discomfort, loss of consciousness, brain damage or death. Diabetes patients are thus hesitant to treat their high or moderately high blood sugar values aggressively out of fear for hypoglycaemia. It could help diabetes treatment if insulin drugs were developed that were only active or released from a depot at higher blood glucose values and were inactive or weakly active at lower glucose values. WO2020201041 discloses glucose sensitive insulin derivates with aryl boron containing moieties displaying glucose-sensitive albumin binding. These insulin derivatives thus display insulin activity dependent on the glucose concentration. To allow for less frequent administration there is a need for glucose sensitive insulin derivatives with longer half-life.

### SUMMARY

In the broadest aspect, the present invention relates to insulin derivatives. These insulin derivatives activate the insulin receptor and thus lowers blood glucose dependent on the glucose concentration, and thus serve as glucose sensitive insulin derivatives.

The insulin derivatives of the present invention bind to both glucose and albumin (Human Serum Albumin, HSA), and the HSA affinity is dependent on the concentration of glucose, i.e. the HSA affinity is glucose sensitive. The apparent human insulin receptor (HIR) affinity in presence of HSA thus also become glucose sensitive. The fraction of insulin that is HSA-bound is shielded from binding to the HIR, but glucose-induced release from HSA increases the free fraction of insulin, and glucose thus increases the HIR apparent affinity. In the presence of HSA, as e.g. in blood, the insulin derivatives of the present invention thus display higher apparent insulin receptor affinity in presence of glucose than when no glucose is present. The insulin derivatives of the present invention are thus glucose sensitive. The insulin derivatives of the present invention display high glucose sensitivity and has a long half-life.

In one aspect, an insulin derivative of the invention comprises an insulin peptide comprising a Lys residue in position 29 of the B-chain of the insulin peptide (B29K); a peptide extension at the N-terminal of the insulin peptide B-chain; and three modifying groups, wherein one modifying group is attached to a Lys residue in position 29 of the B-chain of the insulin peptide, and the two other modifying groups are attached to Lys residues in the peptide extension.

In one aspect, the peptide extension has the sequence of

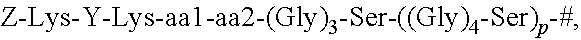

wherein Z consists of 1 to 5 amino acid residues independently selected from Gly, Glu, Ala, Ser, Thr, Pro, and Gln,
wherein Y consists of 15 to 30 amino acid residues independently selected from Gly, Glu, Ala, Ser, Thr, Pro, and Gln,
wherein aa1 is absent or Pro,
wherein aa2 is Glu or Gly,
wherein p is 1, 2, or 3, and
wherein # denotes the attachment point to the N-terminal of the B-chain of human insulin or human insulin analogue.

In one aspect, the modifying groups are of Formula A:

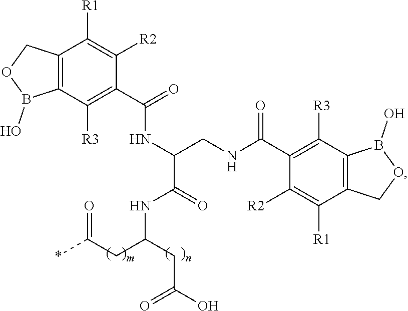

Wherein one of R1, R2, or R3 is an electron withdrawing group, and the two others are hydrogen;
wherein m is 0, 1, 2, or 3; and wherein n is 0, 1, 2, or 3. * denotes the attachment point.

In one aspect, the invention relates to a pharmaceutical composition comprising an insulin derivative according to the invention. In one aspect, the invention relates to an insulin derivative according to the invention for use as a medicament. In one aspect, the invention relates to an insulin derivative according to the invention for use in the treatment of diabetes.

In one aspect, the invention relates to medical use(s) of an insulin derivative according to the invention.

In one aspect, the insulin derivatives of the present invention display high glucose sensitivity.

In one aspect, the insulin derivatives of the present invention have a long half-life. In one aspect, the insulin derivatives of the present invention display high glucose sensitivity and have a long half-life.

The invention may also solve further problems that will be apparent from the disclosure of the exemplary embodiments.

### BRIEF DESCRIPTION OF DRAWINGS

FIG. 1 depicts concentration-response curves for insulin receptor phosphorylation in human primary hepatocytes for the insulin derivative of Example 4 and human insulin at 3 mM and 20 mM D-glucose, respectively (the data in FIG. 1 is from Example 13).

FIG. 2 depicts a representative concentration-response curves from a lipogenesis assay (rFFC) in adipocytes from Sprague Dawley rat for the insulin derivative of Example 4 at 3 mM and 20 mM L-glucose, respectively (the data in FIG. 2 is from Example 14).

FIG. 3 depicts concentration-response curves for glycogen accumulation in rat primary hepatocytes for the insulin derivative of Example 4 and human insulin at 20 mM D-glucose (the data in FIG. 3 is from Example 15).

### DESCRIPTION

In one aspect, an insulin derivative of the invention comprises an insulin peptide, a peptide extension at the N-terminal of the insulin peptide B-chain, and three modifying groups.

### Insulin Derivative

The term “insulin derivative” as used herein means a modified insulin peptide, wherein the modifications are in the form of attachment of chemical moieties and/or the presence of a peptide extension.

In one aspect, the modification is in the form of covalent attachment of modifying groups of Formula A. In one aspect, the modification is in the form of the presence of a peptide extension at the N-terminal of the insulin peptide B-chain. In one aspect, the modification is in the form of covalent attachment of modifying groups of Formula A and the presence of a peptide extension at the N-terminal of the insulin peptide B-chain.

### Insulin Peptide

The term “insulin peptide” as used herein means a peptide which is either human insulin or a human insulin analogue. In one embodiment, the term “insulin peptide” as used herein means a peptide which is either human insulin or an analogue thereof with insulin activity, i.e., which activates the insulin receptor.

### Human Insulin

The term “human insulin” as used herein means the human insulin hormone whose structure and properties are well-known. Human insulin has two polypeptide chains, named the A-chain and the B-chain. The A-chain is a 21 amino acid peptide and the B-chain is a 30 amino acid peptide, the two chains being connected by disulphide bridges: a first bridge between the cysteine in position 7 of the A-chain and the cysteine in position 7 of the B-chain, and a second bridge between the cysteine in position 20 of the A-chain and the cysteine in position 19 of the B-chain. A third bridge is present between the cysteines in position 6 and 11 of the A-chain. The human insulin A-chain has the following sequence: GIVEQCCTSICSLYQLENYCN (SEQ ID NO:1), while the human insulin B-chain has the following sequence: FVNQHLCGSHLVEALYLVCGERGFFYTPKT (SEQ ID NO:2).

### Insulin Analogue

The term “insulin analogue” as used herein means a modified human insulin wherein one or more amino acid residues of the insulin have been substituted by other amino acid residues and/or wherein one or more amino acid residues have been deleted from the insulin and/or wherein one or more amino acid residues have been added and/or inserted to the insulin. The term “insulin analogue” as used herein means an insulin analogue displaying insulin activity, i.e. which binds to and activates the insulin receptor.

The human insulin analogue comprises less than 10 amino acid modifications (substitutions, deletions, additions (i.e. extensions), insertions, and any combination thereof) relative to human insulin, alternatively less than 9, 8, 7, 6, 5, 4, 3, 2, or 1 modification relative to human insulin. In one aspect, the human insulin analogue has less than 10 amino acid modifications (substitutions, deletions, additions (i.e. extensions), insertions, and any combination thereof) relative to human insulin, alternatively less than 9, 8, 7, 6, 5, 4, 3, 2, or 1 modification relative to human insulin.

Modifications in the insulin molecule are denoted stating the chain (A or B), the position, and the one or three letter code for the amino acid residue substituting the native amino acid residue. Herein terms like “A1”, “A2” and “A3” etc. indicates the amino acid in position 1, 2 and 3 etc., respectively, in the A chain of insulin (counted from the N-terminal end). Similarly, terms like B1, B2 and B3 etc. indicates the amino acid in position 1, 2 and 3 etc., respectively, in the B chain of insulin (counted from the N-terminal end). Using the one letter codes for amino acids, the term B29K designates that the amino acid in the B29 position is K. Using the three letter codes for amino acids, the corresponding expression is B29Lys. By “desB30” is meant an insulin analogue lacking the B30 amino acid.

In particular embodiments, an analogue “has” or “comprises” specified changes. In other particular embodiments, an analogue “consists of” the changes. When the term “consists” or “consisting” is used in relation to an analogue e.g. an analogue consists or consisting of a group of specified amino acid mutations, it should be understood that the specified amino acid mutations are the only amino acid mutations in the analogue. In contrast an analogue “comprising” a group of specified amino acid mutations may have additional mutations.

Examples of insulin analogues include:
desB30 human insulin (A-chain of SEQ ID NO:1 and B-chain of SEQ ID NO:3).

### Peptide Extension

As stated above, the insulin analogue of the invention comprises less than 10 amino acid modifications ((substitutions, deletions, additions (i.e. extensions), insertions, and any combination thereof) relative to human insulin, alternatively less than 9, 8, 7, 6, 5, 4, 3, 2, or 1 modification relative to human insulin. In addition to these up to 9 modifications, the insulin peptide of the present invention has a peptide extension at the N-terminal of the B-chain of the insulin peptide.

Various peptide extensions or spacer peptides are known in the art and may be used in the insulin derivatives of the present invention. In one embodiment, the peptide extension is a peptide segment consisting of 28-58 amino acids connected via peptide bonds. The peptide extension of the present invention comprises one or more of the following amino acid residues: Gly (G), Glu (E), Ala (A), Ser(S), Thr (T), Pro (P), Gln (Q), and Lys (K).

The peptide extension in the insulin derivatives of the present invention comprises two Lys (K) residues to allow for attachment of two modifying groups. The exact position of attachment of the modifying groups may vary, as well as some variability of the exact peptide sequence is allowed.

The peptide extension used in the insulin derivatives of the invention has the following peptide sequence:

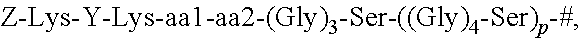

wherein Z consists of 1 to 5 amino acid residues independently selected from Gly, Glu, Ala, Ser, Thr, Pro, and Gln,
wherein Y consists of 15 to 30 amino acid residues independently selected from Gly, Glu, Ala, Ser, Thr, Pro, and Gln,
wherein aa1 is absent or Pro,
wherein aa2 is Glu or Gly,
wherein p is 1, 2, or 3, and
wherein # denotes the attachment point to the N-terminal of the B-chain of the human insulin or human insulin analogue.

In one embodiment, the peptide extension is kept overall polar, for example by having at least five glutamic acid (Glu) amino acid residues in the peptide extension.

When insulin is produced recombinantly, it is expressed as a single-chain insulin precursor which is later matured into 2-chain insulin using proteases, such as Achromobacter lyticus lysyl-specific endoprotease (ALP) or trypsin. ALP specifically and exclusively cleaves lysyl bonds including lysyl-proline bonds. However, as the peptide extension used in the insulin derivatives of the present invention comprises two lysine (Lys) residues, cleavage by ALP can result in undesirable over-cleavage products. It was surprisingly found that by having

Pro-Glu following Lys, the cleavage rate by ALP is greatly reduced, and over-cleavage can thus be reduced, increasing the overall yield.

In one embodiment, the peptide extension used in the insulin derivatives of the invention has the following peptide sequence:

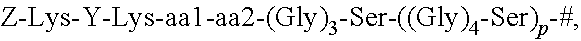

wherein Z consists of 1 to 5 amino acid residues independently selected from Gly, Glu, Ala, Ser, Thr, Pro, and Gln,
wherein Y consists of Pro-Gly followed by 13 to 28 amino acid residues independently selected from Gly, Glu, Ala, Ser, Thr, Pro, and Gln,
wherein aa1 is Pro,
wherein aa2 is Glu,
wherein p is 1, 2, or 3, and
wherein # denotes the attachment point to the N-terminal of the B-chain of the human insulin or human insulin analogue.

More specifically, the peptide extension is attached to the insulin peptide via the carboxylic acid of the C-terminal serine residue of the peptide extension and the amino group of the N-terminal amino acid residue of B-chain of the insulin peptide forming an amide bond between the two amino acid residues.

Examples of peptide extensions at the N-terminal of the B-chain of the insulin peptide include: GKPE(GEQP)₄GEQGGKPEGGGS (G₄S)₂ (SEQ ID NO:4).

### Modifying Group A

The insulin derivatives of the present invention comprise three modifying groups A. In one embodiment, an insulin derivative of the invention has exactly three modifying groups of Formula A. The modifying groups may be identical or different. In one embodiment, the modifying groups are identical. The modifying groups are covalently attached to the epsilon amino group of lysine residues in the insulin peptide and the peptide extension.

The modifying group of the insulin derivatives of the present invention is of Formula A:

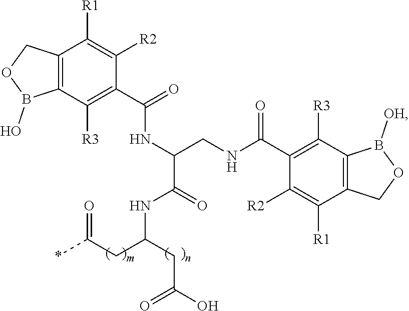

wherein one of R1, R2, or R3 is an electron withdrawing group, and the two others are hydrogen;
wherein m is 0, 1, 2, or 3; and
wherein n is 0, 1, 2, or 3.

Throughout the formulae in the present application, B denotes a boron atom, H denotes a hydrogen atom, N denotes a nitrogen atom, and O denotes an oxygen atom.

In other words, R1, R2 and R3 are independently selected from the group of an electron withdrawing group and hydrogen, with the proviso that if R1 is an electron withdrawing group, then R2 and R3 are hydrogen; if R2 is an electron withdrawing group, then R1 and R3 are hydrogen; and if R3 is an electron withdrawing group, then R1 and R2 are hydrogen. In one embodiment, R1 is an electron withdrawing group, and R2 and R3 are hydrogen.

In order to adjust the pKa of the boronic acids and benzoxaboroles to facilitate binding of glucose at neutral pH, an electron withdrawing group is attached.

An electron withdrawing group (EWG) is an atom or a group that draws electron density from neighbouring atoms towards itself. In one embodiment, the electron withdrawing group is selected from the group of CF₃, F, NO₂, CN, COX, SO₂X, and POX₂, wherein X is OR or NR₂, and R is independently selected from H, alkyl and aryl. In one embodiment, the electron withdrawing group is selected from the group of CF₃, F, NO₂, CN, COX, and SO₂X, wherein X is OR or NR₂, and R is independently selected from H, alkyl and aryl. In one embodiment, the electron withdrawing group is CF₃, F, SO₂NR₂, or CN, wherein R is alkyl. In one embodiment, the electron withdrawing group is CF₃. In one embodiment, R1 is CF₃ and R2 and R3 are hydrogen.

In the insulin derivatives of the present invention, one modifying group is attached to the lysine (Lys) residue in position 29 of the B-chain of the insulin peptide, and the two other modifying groups are attached to each of the lysine (Lys) residues in the peptide extension. More specifically, each modifying group is attached to the epsilon amino group of the lysine forming an amide bond.

Formula A is covalently attached to the Lys via the attachment point shown with *.

The modifying group of Formula A has two chiral centres. Each of the chiral atoms in the modifying group of Formula A can independently be of the (R)- or (S)-form. In one embodiment, both chiral atoms are of the (S)-form. In yet another embodiment, the chiral centres are as shown in Formula A1:

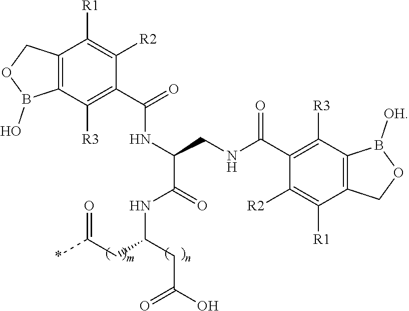

R1, R2, R3, m and n are as defined above for Formula A.

In a particular embodiment, the modifying group is of Formula A, wherein R1 is CF₃, R2 and R3 are hydrogen, m is 2 and n is 0.

In a particular embodiment, the modifying group is of Formula A1, wherein R1 is CF₃, R2 and R3 are hydrogen, m is 2 and n is 0.

### Function of the Insulin Derivatives of the Present Invention

It was surprisingly found that the insulin derivatives of the present invention display both long half-life and high glucose sensitivity in the presence of HSA.

The relative binding affinity for the human insulin receptor (HIR) of insulin analogues and insulin derivatives can be determined by competition binding in a scintillation proximity assay (SPA) as described in Example 12.

In one embodiment, the insulin derivatives of the invention have the ability to bind to the insulin receptor. In one embodiment, the insulin derivatives of the invention have higher apparent insulin receptor affinity in presence of HSA and 20 mM glucose than when no glucose is present. The increase in apparent relative affinity from 0 to 20 mM glucose (HIR glucose factor) in the presence of HSA reflects the glucose sensitivity of an insulin derivative. The glucose factor is above 1 when the relative insulin receptor affinity is higher in the presence of 20 mM glucose, as compared to when no glucose is present. In one embodiment, the insulin derivatives of the present invention have a glucose factor of at least 20, 30, or 40 in presence of 1.5% HSA.

As can be seen from data shown in Example 12, it was found that a peptide extension at the N-terminal of the B-chain of the insulin backbone results in a decrease in glucose sensitivity in the presence of HSA, as measured by the fold change in affinity from 0 to 20 mM glucose (glucose factor). It was surprisingly found that the insulin derivatives of the present invention display high glucose sensitivity in presence of HSA.

The insulin receptor phosphorylation assay described in Example 13, the lipogenesis assay described in Example 14 and the glycogen accumulation assay described in Example 15 can be used as a measure of the functional (agonistic) activity of an insulin derivative. As can be seen from data shown in Example 13, 14, and 15, the insulin derivatives of the present invention activate the insulin receptor. The half-life (T½) upon subcutaneous administration to LYD pigs can be determined using the method as described in Example 16. As can be seen from the pharmacokinetic (PK) studies in LYD pig in Example 16, the compounds of the present invention show a long half-life (T½).

### Intermediate Products

The invention furthermore provides an intermediate product in the form of an insulin analogue having a peptide extension at the N-terminal of the insulin peptide.

Examples of insulin analogues having a peptide extension at the N-terminal of the B-chain of the insulin peptide include:

GKPE(GEQP)₄GEQGGKPEGGGS (G₄S)₂-B1 desB30 human insulin (A-chain of SEQ ID NO: 1 and B-chain of SEQ ID NO:5).

GKPE(GEQP)₄GEQGGKPEGGGS (G₄S)₂-B1 desB30 human insulin means desB30 human insulin extended from B1 with GKPE(GEQP)₄GEQGGKPEGGGS (G₄S)₂. The C-terminal serine(S) is connected to phenylalanine (F) in position B1 of desB30 human insulin.

### General Definitions

The term “compound” is used herein to refer to a molecular entity, and “compounds” may thus have different structural elements besides the minimum element defined for each compound or group of compounds. The term “compound” is also meant to cover pharmaceutically relevant forms hereof, i.e. the invention relates to a compound as defined herein or a pharmaceutically acceptable salt, amide, or ester thereof.

The term “peptide” or “polypeptide”, as e.g. used in the context of the invention, refers to a compound which comprises a series of amino acids interconnected by amide (or peptide) bonds. In a particular embodiment the peptide consists of amino acids interconnected by peptide bonds.

The term “amino acid” includes proteinogenic (or natural) amino acids (amongst those the 20 standard amino acids), as well as non-proteinogenic (or non-natural) amino acids.

Proteinogenic amino acids are those which are naturally incorporated into proteins. The standard amino acids are those encoded by the genetic code. Non-proteinogenic amino acids are either not found in proteins, or not produced by standard cellular machinery (e.g., they may have been subject to post-translational modification).

In general, amino acid residues (peptide/protein sequences) may be identified by their full name, their one-letter code, and/or their three-letter code. These three ways are fully equivalent. Throughout this application, each amino acid of the insulin peptide and the peptide extension of the invention for which the optical isomer is not stated is to be understood to mean the L-isomer (unless otherwise specified). Amino acids are molecules containing an amino group and a carboxylic acid group, and, optionally, one or more additional groups, often referred to as a side chain.

Herein, the term “amino acid residue” is an amino acid from which, formally, a hydroxy group has been removed from a carboxy group and/or from which, formally, a hydrogen atom has been removed from an amino group.

The term “alkyl” as used herein is a branched or unbranched saturated hydrocarbon group of 1 to 24 carbon atoms. Examples include, but are not limited to, methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, and tetradecyl.

The term “aryl” means a cyclic or polycyclic aromatic ring having from 5 to 12 carbon atoms. Examples of aryl groups include, but are not limited to, phenyl, biphenyl, and naphthyl.

The terms “comprise” or “comprising” certain features are to be interpreted as meaning that the subject matter in question includes those certain features, but that it does not exclude the presence of other features.

### Pharmaceutical Composition

The invention also relates to pharmaceutical compositions comprising an insulin derivative of the invention, or a pharmaceutically acceptable salt, amide, or ester thereof, and one or more pharmaceutically acceptable excipient(s). Such compositions may be prepared as is known in the art.

The term “excipient” broadly refers to any component other than the active therapeutic ingredient(s). The excipient may be an inert substance, an inactive substance, and/or a not medicinally active substance. The excipient may serve various purposes, e.g. as a carrier, vehicle, diluent, and/or to improve administration, and/or absorption of the active substance. Non-limiting examples of excipients are solvents, diluents, buffers, preservatives, tonicity regulating agents, chelating agents, and stabilisers. The formulation of pharmaceutically active ingredients with various excipients is known in the art, see e.g. Remington: The Science and Practice of Pharmacy (e.g. 21st edition (2005), and any later editions). A composition of the invention may be in the form of a liquid formulation, i.e. aqueous formulation comprising water. A liquid formulation may be a solution, or a suspension. A composition of the invention may be for parenteral administration, e.g. performed by subcutaneous, intramuscular, intraperitoneal, or intravenous injection.

Aryl boron compounds such as aryl boronic acids may have low stability in aqueous solutions at pH near neutral value. The insulin derivatives of the present invention show improved stability in aqueous solution. Stability can for instance be assessed by measuring the purity of the insulin derivatives after standing in aqueous solution at neutral pH at 25° or 37° Celsius for an extended period of time, for instance a week.

### Pharmaceutical Indications

### Diabetes

The term “diabetes” or “diabetes mellitus” includes type 1 diabetes, type 2 diabetes, gestational diabetes (during pregnancy) and other states that cause hyperglycaemia. The term is used for a metabolic disorder in which the pancreas produces insufficient amounts of insulin, or in which the cells of the body fail to respond appropriately to insulin thus preventing cells from absorbing glucose. As a result, glucose builds up in the blood. Type 1 diabetes, also called insulin-dependent diabetes mellitus (IDDM) and juvenile-onset diabetes, is caused by beta-cell destruction, usually leading to absolute insulin deficiency. Type 2 diabetes, also known as non-insulin-dependent diabetes mellitus (NIDDM) and adult-onset diabetes, is associated with predominant insulin resistance and thus relative insulin deficiency and/or a predominantly insulin secretory defect with insulin resistance.

In one embodiment, an insulin derivative according to the invention is used as a medicament for the treatment or prevention of diabetes, diabetes of Type 1, diabetes of Type 2, impaired glucose tolerance, hyperglycemia, and metabolic syndrome such as metabolic syndrome X or insulin resistance syndrome. In one embodiment, an insulin derivative according to the invention is used as a medicament for the treatment or prevention of hyperglycaemia including stress induced hyperglycemia, type 2 diabetes, impaired glucose tolerance, or type 1 diabetes. In another embodiment, an insulin derivative according to the invention is used as a medicament for delaying or preventing disease progression in type 2 diabetes. The term “treatment” is meant to include both the prevention and minimization of the referenced disease, disorder, or condition (i.e., “treatment” refers to both prophylactic and therapeutic administration of an insulin derivative of the present invention or a composition comprising an insulin derivative of the present invention unless otherwise indicated or clearly contradicted by context).

### Mode of Administration

The route of administration may be any route which effectively transports an insulin derivative of this invention to the desired or appropriate place in the body, such as parenterally, for example, subcutaneously, intramuscularly or intravenously.

For parenteral administration, an insulin derivative of this invention is formulated analogously with the formulation of known insulins. Furthermore, for parenteral administration, an insulin derivative of this invention may be administered analogously with the administration of known insulins and the physicians are familiar with this procedure.

The amount of an insulin derivative of this invention to be administered, the determination of how frequently to administer an insulin derivative of this invention, and the election of which compound or compounds of this invention to administer, optionally together with another antidiabetic compound, is decided in consultation with a practitioner who is familiar with the treatment of diabetes.

### Non-Limiting Embodiments

The invention is further described by the following non-limiting embodiments of the invention:
1. An insulin derivative comprising
   A. an insulin peptide comprising B29K;
   B. a peptide extension Z-Lys-Y-Lys-aa1-aa2-(Gly)₃-Ser-((Gly)₄-Ser)ₚ-#,
   wherein Z consists of 1 to 5 amino acid residues independently selected from Gly,
   Glu, Ala, Ser, Thr, Pro, and Gln,
   wherein Y consists of 15 to 30 amino acid residues independently selected from Gly, Glu, Ala, Ser, Thr, Pro, and Gln,
   wherein aa1 is absent or Pro,
   wherein aa2 is Glu or Gly,
   wherein p is 1, 2, or 3, and
   wherein # denotes the attachment point to the N-terminal of the B-chain of the human insulin or human insulin analogue;
   C. three modifying groups of Formula A:

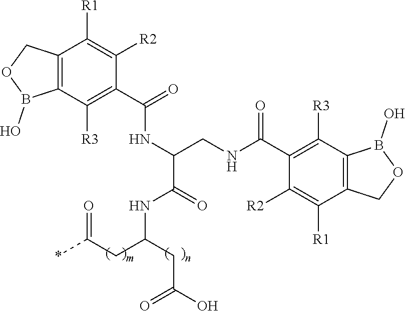

wherein one of R1, R2, or R3 is an electron withdrawing group, and the two others are hydrogen;
wherein m is 0, 1, 2, or 3;
wherein n is 0, 1, 2, or 3;
wherein * denotes the point of attachment and
wherein one modifying group is attached to the Lys residue in position 29 of the B-chain of the insulin peptide, and the two other modifying groups are attached to each of the Lys residues in the peptide extension.
2. The insulin derivative according to the preceding embodiment, wherein Z consists of 1 to 4 amino acid residues independently selected from Gly, Glu, Ala, Ser, Thr, Pro, and Gln.
3. The insulin derivative according to any one of the preceding embodiments, wherein Z consists of 1 to 3 amino acid residues independently selected from Gly, Glu, Ala, Ser, Thr, Pro, and Gln.
4. The insulin derivative according to any one of the preceding embodiments, wherein Z consists of 1 to 2 amino acid residues independently selected from Gly, Glu, Ala, Ser, Thr, Pro, and Gln.
5. The insulin derivative according to any one of the preceding embodiments, wherein Z consists of 1 amino acid residue independently selected from Gly, Glu, Ala, Ser, Thr, Pro, and Gln.
6. The insulin derivative according to any one of the preceding embodiments, wherein Z consists of 1 to 4 Gly amino acid residues.
7. The insulin derivative according to any one of the preceding embodiments, wherein Z consists of 1 to 3 Gly amino acid residues.
8. The insulin derivative according to any one of the preceding embodiments, wherein Z consists of 1 to 2 Gly amino acid residues.
9. The insulin derivative according to any one of the preceding embodiments, wherein Z is Gly.
10. The insulin derivative according to any one of the preceding embodiments, wherein Y consists of 20 to 25 amino acid residues independently selected from Gly, Glu, Ala, Ser, Thr, Pro, and Gln.
11. The insulin derivative according to any one of the preceding embodiments, wherein Y consists of 23 amino acid residues independently selected from Gly, Glu, Ala, Ser, Thr, Pro, and Gln.
12. The insulin derivative according to any one of the preceding embodiments, wherein Y consists of 15 to 30 amino acid residues independently selected from Gly, Glu, Pro, and Gln.
13. The insulin derivative according to any one of the preceding embodiments, wherein Y consists of 20 to 25 amino acid residues independently selected from Gly, Glu, Pro, and Gln.
14. The insulin derivative according to any one of the preceding embodiments, wherein Y consists of 23 amino acid residues independently selected from Gly, Glu, Pro, and Gln.
15. The insulin derivative according to any one of the preceding embodiments, wherein Y consists of 15 to 30 amino acid residues independently selected from Gly, Ala, Pro, and Gln.
16. The insulin derivative according to any one of the preceding embodiments, wherein Y consists of 20 to 25 amino acid residues independently selected from Gly, Ala, Pro, and Gln.
17. The insulin derivative according to any one of the preceding embodiments, wherein Y consists of 23 amino acid residues independently selected from Gly, Ala, Pro, and Gln.
18. The insulin derivative according to any one of the preceding embodiments, wherein Y consists of Pro-Gly followed by 18 to 23 amino acid residues independently selected from Gly, Glu, Ala, Ser, Thr, Pro, and Gln.
19. The insulin derivative according to any one of the preceding embodiments, wherein Y consists of Pro-Gly followed by 20 to 22 amino acid residues independently selected from Gly, Glu, Ala, Ser, Thr, Pro, and Gln.
20. The insulin derivative according to any one of the preceding embodiments, wherein Y consists of Pro-Gly followed by 21 amino acid residues independently selected from Gly, Ala, Ser, Thr, Pro, and Gln.
21. The insulin derivative according to any one of the preceding embodiments, wherein Y consists of Pro-Gly followed by 18 to 23 amino acid residues independently selected from Gly, Glu, Pro, and Gln.
22. The insulin derivative according to any one of the preceding embodiments, wherein Y consists of Pro-Gly followed by 20 to 22 amino acid residues independently selected from Gly, Glu, Pro, and Gln.
23. The insulin derivative according to any one of the preceding embodiments, wherein Y consists of Pro-Gly followed by 21 amino acid residues independently selected from Gly, Glu, Pro, and Gln.
24. The insulin derivative according to any one of the preceding embodiments, wherein Y is PE(GEQP)₄GEQGG (SEQ ID NO:13).
25. The insulin derivative according to any one of the preceding embodiments, wherein aa1 is Pro.
26. The insulin derivative according to any one of the preceding embodiments, wherein aa2 is Glu.
27. The insulin derivative according to any one of the preceding embodiments, wherein p is 2.
28. The insulin derivative according to any one of the preceding embodiments, wherein the peptide extension is GKPE(GEQP)₄GEQGGKPEGGGS (G₄S)₂ (SEQ ID NO:4).
29. The insulin derivative according to any one of the preceding embodiments, wherein the modifying group is of Formula A1:

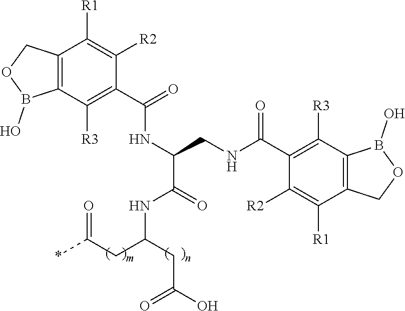

wherein one of R1, R2, or R3 is an electron withdrawing group, and the two others are hydrogen;
wherein m is 0, 1, 2, or 3;
wherein n is 0, 1, 2, or 3; and
   wherein * denotes the point of attachment.
30. The insulin derivative according to any one of the preceding embodiments, wherein the electron withdrawing group is selected from the group of CF₃, F, NO₂, CN, COX, SO₂X, and POX₂, wherein X is OR or NR₂, and R is H, alkyl or aryl.
31. The insulin derivative according to any one of the preceding embodiments, wherein the electron withdrawing group is selected from the group of CF₃, F, NO₂, CN, COX, and SO₂X, wherein X is OR or NR₂, and R is H, alkyl or aryl.
32. The insulin derivative according to any one of the preceding embodiments, wherein X is OR.
33. The insulin derivative according to any one of the preceding embodiments, wherein X is NR₂.
34. The insulin derivative according to any one of the preceding embodiments, wherein R is H.
35. The insulin derivative according to any one of the preceding embodiments, wherein R is alkyl.
36. The insulin derivative according to any one of the preceding embodiments, wherein alkyl is selected from the group of methyl, ethyl, n-propyl, and isopropyl.
37. The insulin derivative according to any one of the preceding embodiments, wherein alkyl is selected from the group of methyl and ethyl.
38. The insulin derivative according to any one of the preceding embodiments, wherein alkyl is methyl.
39. The insulin derivative according to any one of the preceding embodiments, wherein R is aryl.
40. The insulin derivative according to any one of the preceding embodiments, wherein aryl is phenyl.
41. The insulin derivative according to any one of the preceding embodiments, wherein the electron withdrawing group is selected from the group of CF₃, F, SO₂NR₂, or CN, wherein R is alkyl.
42. The insulin derivative according to any one of the preceding embodiments, wherein the electron withdrawing group is selected from the group of CF₃, and F.
43. The insulin derivative according to any one of the preceding embodiments, wherein the electron withdrawing group is CF₃.
44. The insulin derivative according to any one of the preceding embodiments, wherein R1 is an electron withdrawing group and R2 and R3 are hydrogen.
45. The insulin derivative according to any one of the preceding embodiments, wherein R1 is CF₃ and R2 and R3 are hydrogen.
46. The insulin derivative according to any one of the preceding embodiments, wherein m is 0, 1, 2, or 3 and n is 0.
47. The insulin derivative according to any one of the preceding embodiments, wherein m is 1, 2, or 3 and n is 0.
48. The insulin derivative according to any one of the preceding embodiments, wherein m is 3 and n is 0.
49. The insulin derivative according to any one of the preceding embodiments, wherein m is 2 and n is 0.
50. The insulin derivative according to any one of the preceding embodiments, wherein m is 1 and n is 0.
51. The insulin derivative according to any one of the preceding embodiments, wherein m is 0 and n is 3.
52. The insulin derivative according to any one of the preceding embodiments, wherein m is 0 and n is 2.
53. The insulin derivative according to any one of the preceding embodiments, wherein m is 0 and n is 1.
54. The insulin derivative according to any one of the preceding embodiments, wherein both chiral atoms in Formula A are of the(S)-form.
55. The insulin derivative according to any one of the preceding embodiments, wherein the insulin peptide is human insulin.
56. The insulin derivative according to any one of the preceding embodiments, wherein the insulin peptide is a human insulin analogue.
57. The insulin derivative according to any one of the preceding embodiments, wherein the insulin peptide is a human insulin analogue comprising less than 10 amino acid modifications relative to human insulin.
58. The insulin derivative according to any one of the preceding embodiments, wherein the insulin peptide is a human insulin analogue comprising less than 9 amino acid modifications relative to human insulin.
59. The insulin derivative according to any one of the preceding embodiments, wherein the insulin peptide is a human insulin analogue comprising less than 8 amino acid modifications relative to human insulin.
60. The insulin derivative according to any one of the preceding embodiments, wherein the insulin peptide is a human insulin analogue comprising less than 7 amino acid modifications relative to human insulin.
61. The insulin derivative according to any one of the preceding embodiments, wherein the insulin peptide is a human insulin analogue comprising less than 6 amino acid modifications relative to human insulin.
62. The insulin derivative according to any one of the preceding embodiments, wherein the insulin peptide is a human insulin analogue comprising less than 5 amino acid modifications relative to human insulin.
63. The insulin derivative according to any one of the preceding embodiments, wherein the insulin peptide is a human insulin analogue comprising less than 4 amino acid modifications relative to human insulin.
64. The insulin derivative according to any one of the preceding embodiments, wherein the insulin peptide is a human insulin analogue comprising less than 3 amino acid modifications relative to human insulin.
65. The insulin derivative according to any one of the preceding embodiments, wherein the insulin peptide is a human insulin analogue comprising less than 2 amino acid modifications relative to human insulin.
66. The insulin derivative according to any one of the preceding embodiments, wherein the insulin peptide is a human insulin analogue comprising desB30.
67. The insulin derivative according to any one of the preceding embodiments, wherein the insulin peptide is desB30 human insulin.
68. The insulin derivative according to any one of the preceding embodiments,
wherein the insulin peptide is desB30 human insulin,
   wherein the peptide extension is Z-Lys-Y-Lys-Pro-Glu-(Gly)₃-Ser-((Gly)₄-Ser)ₚ-#,
   wherein Z consists of 1 to 5 amino acid residues independently selected from Gly, Glu, Pro, and Gln,
   wherein Y consists of Lys-Pro followed by 13 to 23 amino acid residues independently selected from Gly, Glu, Pro, and Gln,
   wherein p is 1, 2, or 3;
   wherein # denotes the attachment point to the N-terminal of the B-chain of the human insulin or human insulin analogue; and
wherein R1 is CF₃, R2 and R3 are hydrogen, m is 2 and n is 0.
69. The insulin derivative according to any one of the preceding embodiments, wherein Z consists of 1 to 3 amino acid residues independently selected from Gly, Glu, Pro, and Gln.
70. The insulin derivative according to any one of the preceding embodiments, wherein Z consists of Gly optionally followed by 1 to 2 amino acid residues independently selected from Gly, Glu, Pro, and Gln.
71. The insulin derivative according to any one of the preceding embodiments, wherein Z is Gly.
72. The insulin derivative according to any one of the preceding embodiments, wherein Y consists of Lys-Pro followed by 20 to 25 amino acid residues independently selected from Gly, Glu, Pro, and Gln.
73. The insulin derivative according to any one of the preceding embodiments, wherein p is 2.
74. The insulin derivative according to any one of the preceding embodiments wherein the insulin peptide has the ability to bind to the insulin receptor.
75. The insulin derivative according to any one of the preceding embodiments, wherein the insulin peptide has the ability to bind to and activate the insulin receptor.
76. The insulin derivative according to any one of the preceding embodiments, wherein the insulin derivative has the ability to bind to the insulin receptor.
77. The insulin derivative according to any one of the preceding embodiments, wherein the insulin derivative has the ability to activate the insulin receptor.
78. The insulin derivative according to any one of the preceding embodiments, wherein the insulin derivative has higher apparent insulin receptor affinity in presence of 20 mM glucose and human serum albumin (HSA) than when no glucose is present.
79. The insulin derivative according to any one of the preceding embodiments, wherein the insulin derivative has at least 20-fold higher apparent insulin receptor affinity in presence of 20 mM glucose and 1.5% human serum albumin (HSA) than when no glucose is present.
80. The insulin derivative according to any one of the preceding embodiments, wherein the insulin derivative has at least 30-fold higher apparent insulin receptor affinity in presence of 20 mM glucose and 1.5% human serum albumin (HSA) than when no glucose is present.
81. The insulin derivative according to any one of the preceding embodiments, wherein the insulin derivative has at least 40-fold higher apparent insulin receptor affinity in presence of 20 mM glucose and 1.5% human serum albumin (HSA) than when no glucose is present.
82. The insulin derivative according to any one of the preceding embodiments, wherein the insulin derivative has from 20-fold to 80-fold higher apparent insulin receptor affinity in presence of 20 mM glucose and 1.5% human serum albumin (HSA) than when no glucose is present.
83. The insulin derivative according to any one of the preceding embodiments, wherein the insulin derivative has from 30-fold to 60-fold higher apparent insulin receptor affinity in presence of 20 mM glucose and 1.5% human serum albumin (HSA) than when no glucose is present.
84. The insulin derivative according to any one of the preceding embodiments, wherein the insulin derivative has from 40-fold to 60-fold higher apparent insulin receptor affinity in presence of 20 mM glucose and 1.5% human serum albumin (HSA) than when no glucose is present.
85. The insulin derivative according to any one of the preceding embodiments, wherein the insulin derivative has from 50-fold to 60-fold higher apparent insulin receptor affinity in presence of 20 mM glucose and 1.5% human serum albumin (HSA) than when no glucose is present.
86. The insulin derivative according to any one of the preceding embodiments, wherein the insulin receptor affinity is determined by the assay described in Example 12.
87. The insulin derivative according to any one of the preceding embodiments, wherein the insulin derivative has a T½ of at least 15 hours upon subcutaneous administration to a LYD pig.
88. The insulin derivative according to any one of the preceding embodiments, wherein the insulin derivative has a T½ of at least 16 hours upon subcutaneous administration to a LYD pig.
89. The insulin derivative according to any one of the preceding embodiments, wherein the insulin derivative has a T½ of at least 17 hours upon subcutaneous administration to a LYD pig.
90. The insulin derivative according to any one of the preceding embodiments, wherein the insulin derivative has a T½ of at least 18 hours upon subcutaneous administration to a LYD pig.
91. The insulin derivative according to any one of the preceding embodiments, wherein the insulin derivative has a T½ of at least 19 hours upon subcutaneous administration to a LYD pig 92. The insulin derivative according to any one of the preceding embodiments, wherein the insulin derivative has a T½ in the range of 15 to 25 hours upon subcutaneous administration to a LYD pig.
93. The insulin derivative according to any one of the preceding embodiments, wherein the insulin derivative has a T½ in the range of 17 to 23 hours upon subcutaneous administration to a LYD pig.
94. The insulin derivative according to any one of the preceding embodiments, wherein the insulin derivative has a T½ in the range of 18 to 23 hours upon subcutaneous administration to a LYD pig.
95. The insulin derivative according to any one of the preceding embodiments, wherein the T½ is determined by the method described in Example 16.
96. The insulin derivative according to any one of the preceding embodiments, wherein the insulin derivative is the insulin derivative of Example 4.
97. The insulin derivative according to any one of the preceding embodiments, wherein the insulin derivative is

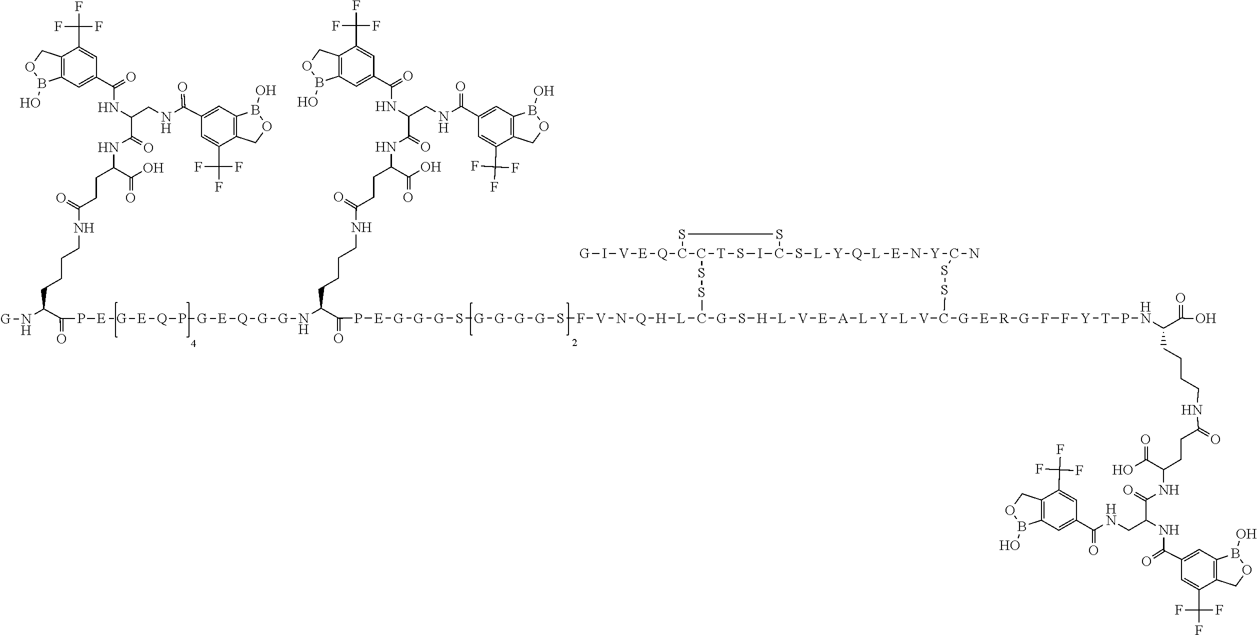

98. The insulin derivative according to any one of the preceding embodiments, wherein the insulin derivative is

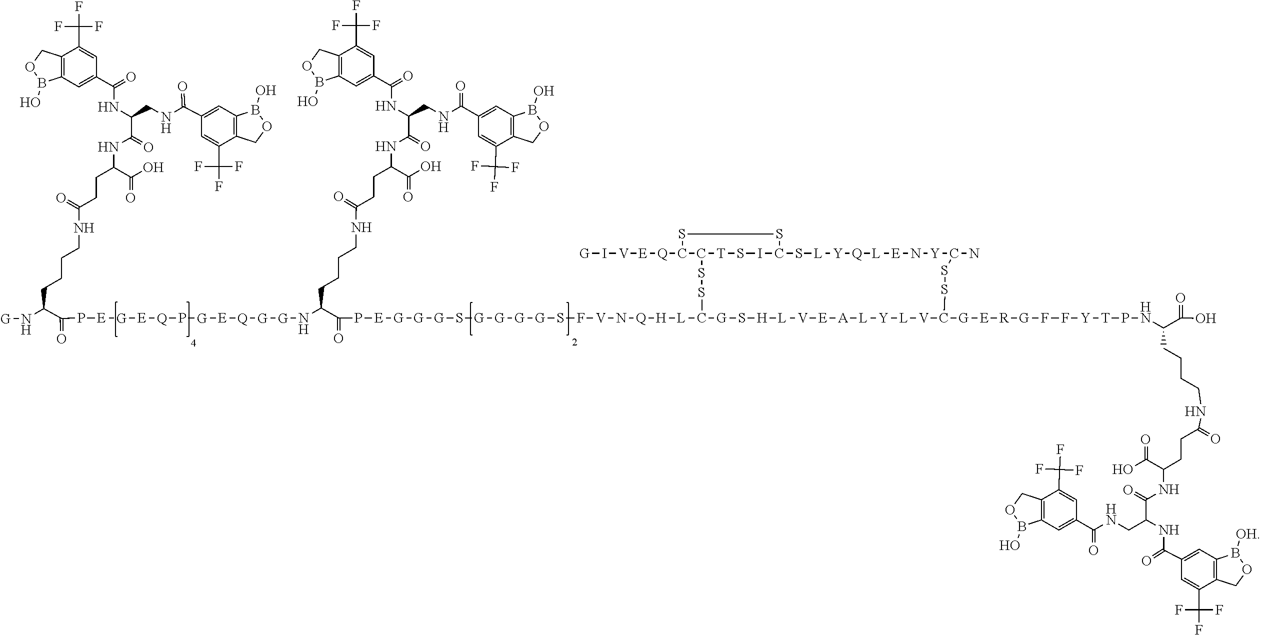

99. The insulin derivative according to any one of the preceding embodiments, wherein the insulin derivative is

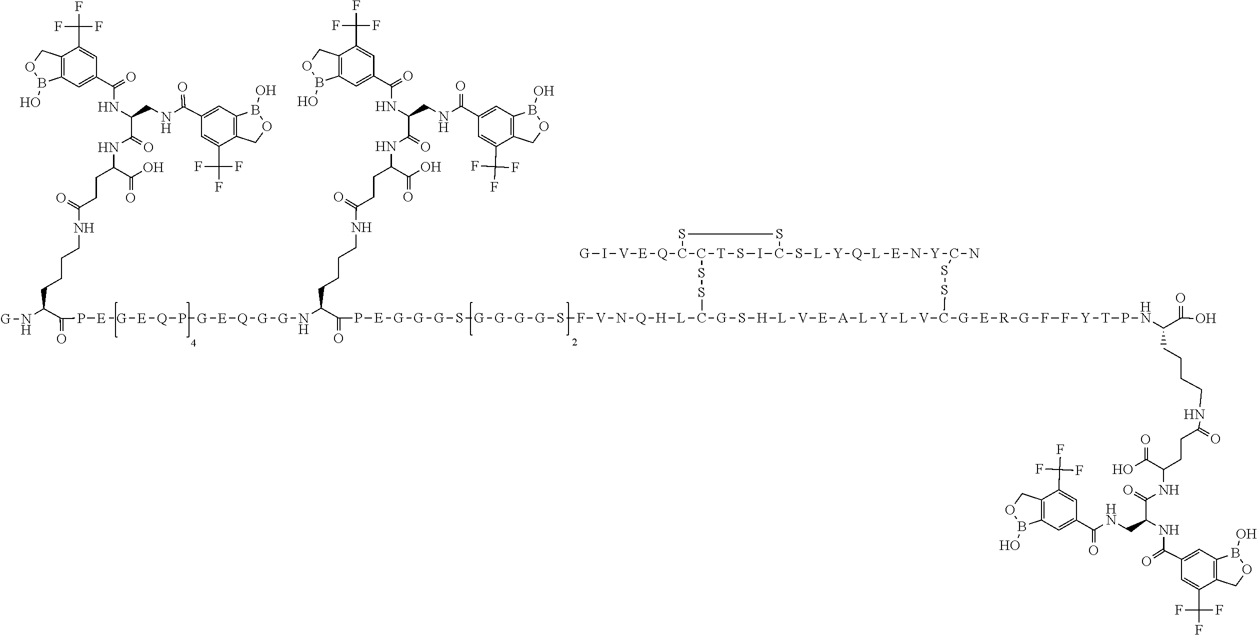

100. The insulin derivative according to any one of the preceding embodiments, wherein the insulin derivative is

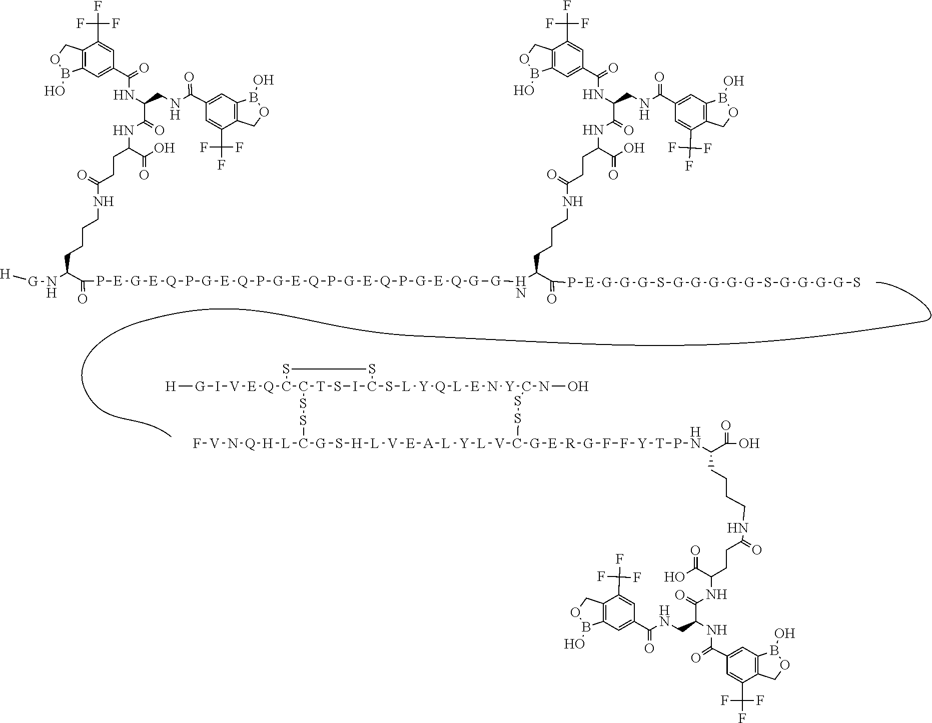

101. The insulin derivative according to any one of the preceding embodiments, wherein the insulin derivative is

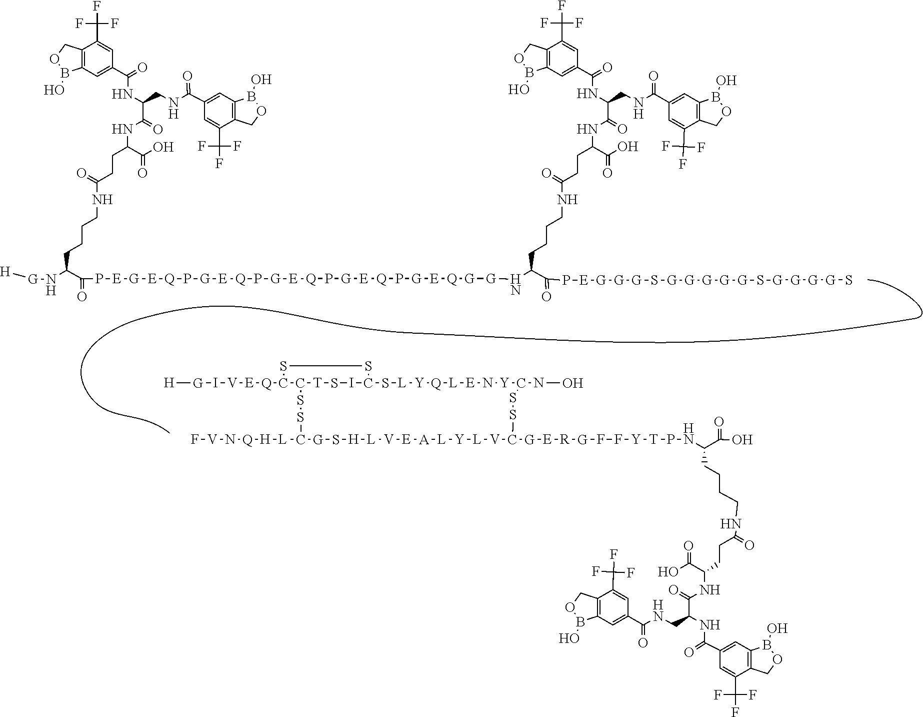

1021. An insulin derivative, wherein the insulin derivative is the insulin derivative of Example 4.
103. An insulin derivative, wherein the insulin derivative is

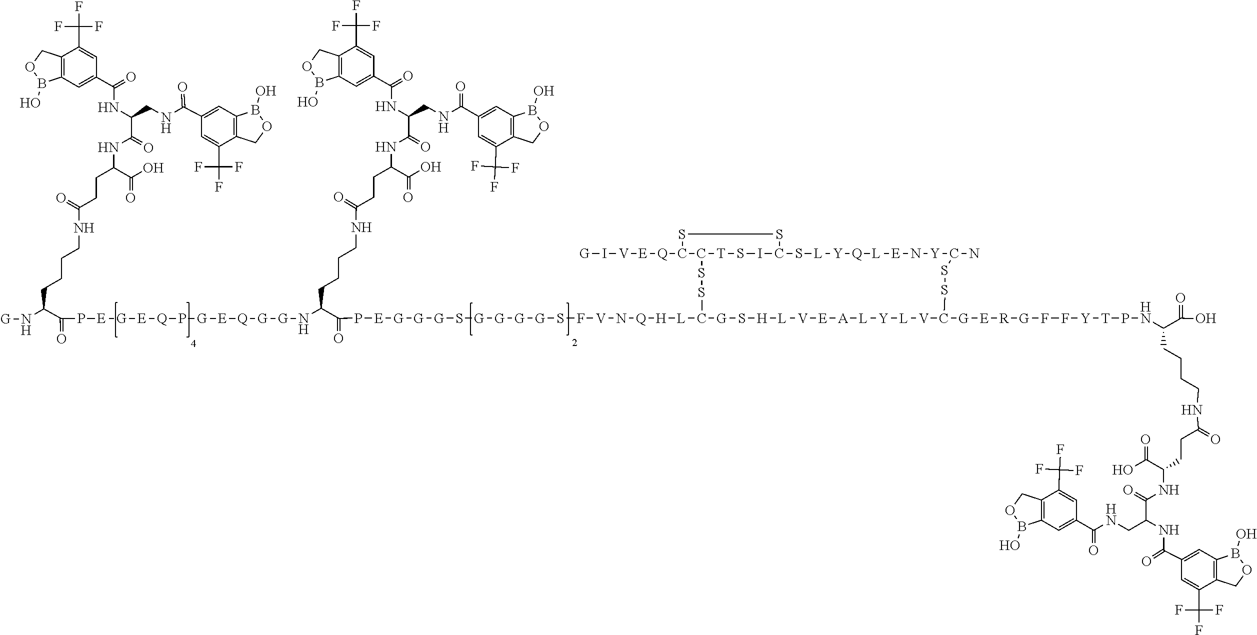

104. A composition comprising an insulin derivative according to any one of embodiments 1 to 103.
105. An insulin derivative according to any one of embodiments 1-103 for use as a medicament.
106. An insulin derivative according to any one of embodiments 1-103 for use in the prevention or treatment of diabetes, diabetes of Type 1, diabetes of Type 2, impaired glucose tolerance, hyperglycemia, and metabolic syndrome, such as metabolic syndrome X or insulin resistance syndrome.
107. An insulin derivative according to any one of embodiments 1-103, for use in the prevention or treatment of diabetes, including diabetes of Type 1 and diabetes of Type 2.
108. An insulin derivative according to any one of embodiments 1 to 103 for use in the prevention or treatment of diabetes.
109. An insulin derivative according to any one of embodiments 1 to 103 for use in the treatment of diabetes.
110. Use of an insulin derivative according to any one of embodiments 1 to 103 or the composition according to embodiment 104, for the manufacture of a medicament for the treatment or prevention of diabetes, diabetes of Type 1, diabetes of Type 2, impaired glucose tolerance, hyperglycemia, and metabolic syndrome, such as metabolic syndrome X or insulin resistance syndrome.
111. Use of an insulin derivative according to any one of embodiments 1 to 103 or the composition according to embodiment 104, for the manufacture of a medicament for the treatment or prevention of diabetes, including diabetes of Type 1 and diabetes of Type 2.
112. Use of an insulin derivative according to any one of embodiments 1 to 103 or the composition according to embodiment 104, for the manufacture of a medicament for the treatment or prevention of diabetes.
113. A method for the treatment or prevention of diabetes, diabetes of Type 1, diabetes of Type 2, impaired glucose tolerance, hyperglycemia, and metabolic syndrome, such as metabolic syndrome X or insulin resistance syndrome, which method comprises administration to a subject in need thereof a therapeutically effective amount of an insulin derivative according to any one of embodiments 1 to 103 or the composition according to embodiment 104.
114. A method for the treatment or prevention of diabetes, including diabetes of Type 1 and diabetes of Type 2, which method comprises administration to a subject in need thereof a therapeutically effective amount of an insulin derivative according to any one of embodiments 1 to 103 or the composition according to embodiment 104.
115. A method for the treatment or prevention of diabetes, which method comprises administration to a subject in need thereof a therapeutically effective amount of an insulin derivative according to any one of the embodiments 1 to 103 or the composition according to embodiment 104.

### EXAMPLES

The insulin conjugates in the examples are drawn using the standard single letter abbreviations for the amino acids. The sulfur atoms of the cysteine residues are drawn out specifically to illustrate disulfide bridges. Residues that are modified by conjugation are drawn out to show exactly where in the relevant amino acid the modification has taken place. The N-termini of insulin are denoted with small font H-, and the C-termini are denoted with small font-OH, as is standard in peptide chemistry. H-and-OH are not used when a terminal residue is modified by conjugation, in which case the residues are drawn expanded, as explained above.

### Materials and Methods

List of Abbreviations

| | |
|---|---|
| AAs | amino acid residues |
| ALP | Achromobacter lyticus lysyl-specific |
| | endoprotease |
| BHK | Baby hamster kidney |
| C18 | octadecanyl (HPLC column) |
| cpm | counts per minutes |
| CV | column volume |
| EDTA | ethylen-diamin-tetra-acetic acid |
| Dap | 2,3-Diaminopropionic acid |
| DCC | N,N'-dicyclohexylcarbodiimide |
| DIC | N,N'-diisopropylcarbodiimide |
| DMF | N,N-dimethylformamide |
| Fmoc | fluorenylmethoxycarbonyl |
| HATU | 1-[bis(dimethylamino)methylene]-1H- |
| | 1,2,3-triazolo[4,5-b]pyridinium 3-oxid |
| | hexafluorophosphate |
| Hepes | (4-(2-hydroxyethyl)-1-piperazineethanesulfonic |
| | acid) |
| HI | Human Insulin |
| HIR | Human Insulin Receptor |
| HIR-A | Human Insulin Receptor A isoform |
| hrs | hours |
| HSA | Human Serum Albumin |
| iv | intravenously |
| LCMS | liquid chromatography mass spectrometry |
| LYD | Landrace-Yorkshire-Duroc |
| min | minutes |
| MeCN | acetonitrile |
| mm | millimeter |
| mM | millimolar |
| NBS | N-bromosuccinimide |
| NHS | N-hydroxysuccinimide |
| nm | nanometer |
| nM | nanomolar |
| NMR | nuclear magnetic resonance |
| RP-HPLC | reverse-phase high performance liquid |
| | chromatography |
| SC | subcutaneously |
| SPA | Scintillation Proximity Assay |
| tBu | tert-butyl |
| TFA | trifluoroacetic acid |
| THF | tetrahydrofuran |
| UHPLC | ultra high performance liquid chromatography |

### Preparation of Insulin Variants

### Example 1: Expression of Insulin Variants in Yeast and Transformation with ALP Etc

The insulin analogues were expressed in yeast (Saccharomyces cerevisiae) using well-known techniques e.g. as disclosed in WO2017/032798. More specifically, the insulin analogues were expressed as single-chain precursors, which were isolated by ion-exchange capture, and cleaved to the 2-chain insulin analogues by treatment with ALP as described below.

### Capture of the Precursor on SP Sepharose BB:

The yeast supernatant was loaded with a flow of 10-20 CV/hrs onto a column packed with SP Sepharose BB. A wash with 0.1 M citric acid pH 3.5 and a wash with 40% EtOH was performed. The analogue was eluted with 0.2 M sodium acetate pH 5.5/35% EtOH.

ALP digestion:

The solution of single-chain precursor was adjusted to pH 9 and ALP enzyme was added 1:100 (w/w). The reaction was followed on UHPLC. ALP cleavage pool was adjusted to pH 2.5 and diluted 2-fold in order to be prepared for RP-HPLC purification.

RP-HPLC Purification:
Purification was performed by RP-HPLC C18 as below:
Column: 15 μm C18 50×250 mm 200 Å

### Buffers:

A: 0.2% formic acid, 5% EtOH,
B: 0.2% formic acid, 50% EtOH
The gradient: 20-55% B-buffer.
Gradient: 20 CV
Flow 20 CV/hrs
Load g~5 g/L resin
Fractions were analysed by UHPLC, pooled and freeze dried.

Insulin analogues prepared and used in the examples below:

GKPE-(GEQP)₄-GEQGGKPEGGGSGGGGSGGGGS-B1 desB30 human insulin (SEQ ID NO: 1 and SEQ ID NO:5)

(GQEP)₄-GQEGGKPGGGGSGGGGSGGGGS-B1 desB30 human insulin (SEQ ID NO:1 and SEQ ID NO:6) (GQEP)₂₄-GQEGGKPGGGGSGGGGSGGGGS-B1 desB30 human insulin (SEQ ID NO:1 and SEQ ID NO:7)

(GEQP)₄-GEQGGKPEGGGSGGGGSGGGGS-B1 desB30 human insulin (SEQ ID NO:1 and SEQ ID NO:8)

GKPE-(GEQP)₄-GEQG-GKPEGGGSGGGGSGGGGS-B1 desB30 human insulin means desB30 human insulin extended from B1 with GKPE-GEQPGEQPGEQPGEQP-GEQG-GKPEGGGSGGGGSGGGGS (C-terminal S connected to B1F). Similar for the other backbones.

### Preparation of Building Blocks

### Example 2: (S)-4-((S)-2,3-Bis(1-hydroxy-4-(trifluoromethyl)-1,3-dihydrobenzo[c][1.2]oxaborole-6-carboxamido) propanamido)-5-(tert-butoxy)-5-oxopentanoic acid

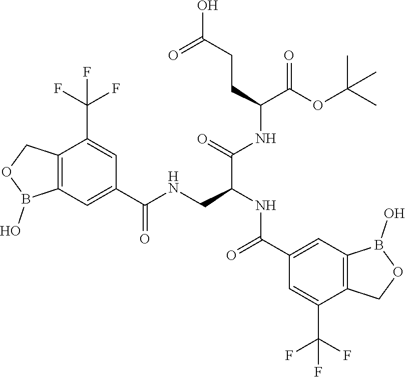 [SMILES: CC(C)(C)OC(=O)[C@H](CCC(=O)O)NC(=O)[C@H](CNC(=O)c1cc2c(c(C(F)(F)F)c1)COB2O)NC(=O)c1cc2c(c(C(F)(F)F)c1)COB2O]

NBS (34.0 g, 191 mmol) was added to a solution of 3-trifluoromethyl-4-methylbenzoic acid (39.0 g, 191 mmol) in concentrated sulfuric acid (400 mL) and the reaction mixture was stirred at room temperature for 16 hrs. The reaction mixture was then poured into ice-water (2 L). Resulting precipitate was filtered off, washed with water (500 mL) and dissolved in ethyl acetate (400 mL); dried over anhydrous sodium sulfate, filtered and evaporated to provide 3-bromo-4-methyl-5-trifluoromethylbenzoic acid as white solid. Yield: 53.4 g (98%). ¹H NMR spectrum (300 MHz, DMSO-d6, δH): 13.71 (bs, 1H); 8.35 (d, J=0.4 Hz, 1H); 8.15 (d, J=0.9 Hz, 1H); 2.56 (s, 3H).

Concentrated sulfuric acid (24 mL) was added to a solution 3-bromo-4-methyl-5-trifluoromethylbenzoic acid (35.0 g, 124 mmol) in methanol (500 mL) and the reaction mixture was allowed to stir under reflux for 4 hrs and at room temperature for 16 hrs. The reaction mixture was then evaporated under reduced pressure, dissolved in diethyl ether (250 mL), washed with water (2×100 mL) and mixture of saturated solution of potassium carbonate (100 mL) and brine (100 mL). Organic layer was separated, dried over anhydrous sodium sulfate, filtered and evaporated to provide methyl 3-bromo-4-methyl-5-trifluoromethylbenzoate as white solid. Yield: 35.3 g (96%). ¹H NMR spectrum (300 MHz, DMSO-d6, δH): 8.36 (d, J=1.1 Hz, 1H); 8.13 (d, J=1.1 Hz, 1H); 3.90 (s, 3H); 2.55 (d, J=1.3 Hz, 3H).

A suspension of NBS (31.7 g, 178 mmol) and methyl 3-bromo-4-methyl-5-trifluoromethylbenzoate (35.3 g, 119 mmol) in water (300 mL) was stirred for 6 hrs under 100 W light bulb at 80° C. Reaction mixture was extracted with diethyl ether (2×200 mL). Organic layers were washed with brine (150 mL). Organic layer was separated, dried over anhydrous sodium sulfate, filtered and evaporated to provide methyl 3-bromo-4-bromomethyl-5-trifluoromethylbenzoate as yellow solid. Yield: 44.0 g (98%). ¹H NMR spectrum (300 MHz, CDCl₃, δH): 8.47 (d, J=1.5 Hz, 1H); 8.31 (d, J=1.3 Hz, 1H); 4.75 (s, 2H); 3.98 (s, 3H).

Solution of methyl 3-bromo-4-bromomethyl-5-trifluoromethylbenzoate (44.0 g, 117 mmol) and potassium acetate (22.9 g, 234 mmol) in MeCN (0.5 L) was stirred at 75° C. overnight. The suspension was filtered through filtering paper and evaporated. The crude product was dissolved in dichloromethane and filtered again. Evaporation provided methyl 4-(acetoxymethyl)-3-bromo-5-(trifluoromethyl)benzoate as white solid. Yield: 37.9 g (91%). ¹H NMR spectrum (300 MHz, CDCl₃, δH): 8.49 (d, J=1.3 Hz, 1H); 8.34 (d, J=1.3 Hz, 1H); 5.37 (s, 2H); 3.99 (s, 3H); 2.11 (s, 3H).

A solution of methyl 4-(acetoxymethyl)-3-bromo-5-(trifluoromethyl)benzoate (37.9 g, 107 mmol), bis(pinacolato)diboron (29.8 g, 117 mmol), potassium acetate (31.4 g, 294 mmol) and [1,1-bis(diphenylphosphino) ferrocene]dichloropalladium (II) (1.57 g, 1.92 mmol) in dry tetrahydrofuran (500 mL) was allowed to stir at 75° C. under argon atmosphere for 13 days. Then the reaction mixture was cooled to room temperature, filtered and evaporated. The crude product was filtered through silica gel column (Silicagel, 0.063-0.200 mm; eluent: cyclohexane/ethyl acetate 8:1) to provide methyl 4-(acetoxymethyl)-3-(4,4,5,5-tetramethyl-1,3,2-dioxaborolan-2-yl)-5-(trifluoromethyl)benzoate. Yield: 31.1 g (72%). ¹H NMR spectrum (300 MHz, CDCl₃, δH): 8.65 (s, 1H); 8.43 (s, 1H); 5.48 (s, 2H); 3.97 (s, 3H); 2.05 (s, 3H); 1.36 (s, 12H).

A solution of methyl 4-(acetoxymethyl)-3-(4,4,5,5-tetramethyl-1,3,2-dioxaborolan-2-yl)-5-(trifluoromethyl)benzoate (31.0 g, 77.1 mmol) and sodium hydroxide (15.4 g, 386 mmol) in water (300 mL) was stirred at room temperature for 3 hrs. Then solution of hydrochloric acid (35 mL) in water (100 mL) was added to lower the pH to 1. The reaction mixture was stirred overnight. Precipitate was filtered off and dried to provide 1-hydroxy-4-(trifluoromethyl)-1,3-dihydrobenzo[c][1,2]oxaborole-6-carboxylic acid as white solid. Yield: 16.6 g (86%).

¹H NMR spectrum (300 MHz, DMSO-d6, δH): 13.47 (bs, 1H); 9.66 (s, 1H); 8.62 (s, 1H); 8.24 (s, 1H); 5.22 (s, 2H).

A solution of pentafluorophenol (7.48 g, 40.7 mmol), 1-hydroxy-4-(trifluoromethyl)-1,3-dihydrobenzo[c][1,2]oxaborole-6-carboxylic acid (10.0 mg, 40.7 mmol) and N,N′-dicyclohexylcarbodiimide (DCC, 8.37 mg, 40.7 mmol) in MeCN (0.5 L) was stirred at room temperature overnight. The reaction mixture was filtered, evaporated, dissolved in MeCN, re-filtered and evaporated to give the pentafluorophenyl 1-hydroxy-4-(trifluoromethyl)-1,3-dihydrobenzo[c][1,2]oxaborole-6-carboxylate as white solid.

Yield: 16.7 g (100%). ¹H NMR spectrum (300 MHz, DMSO-d6, δH): 9.79 (s, 1H); 8.86 (s, 1H); 8.46 (s, 1H); 5.30 (s, 2H).

2-Chlorotrityl chloride resin 100-200 mesh 1.5 mmol/g (3, 4.47 g, 6.71 mmol) was left to swell in dry dichloromethane (30 mL) for 30 min. A solution of (2S)-5-(tert-butoxy)-2-{[(9H-fluoren-9-ylmethoxy) carbonyl]amino}-5-oxopentanoic acid (Fmoc-Glu-OtBu, 1.90 g, 4.47 mmol) and N,N-diisopropylethylamine (2.96 mL, 17.0 mmol) in dry dichloromethane (30 mL) was added to resin and the mixture was shaken overnight. Resin was filtered and treated with a solution of N,N-diisopropylethylamine (1.56 mL, 8.95 mmol) in methanol/dichloromethane mixture (4:1, 2×5 min, 2×40 mL). Then resin was washed with DMF (2×30 mL), dichloromethane (2×40 mL) and DMF (3×40 mL). Fmoc group was removed by treatment with 20% piperidine in DMF (1×5 min, 1×20 min, 2×40 mL). Resin was washed with DMF (3×40 mL), 2-propanol (2×40 mL) and dichloromethane (3×40 mL). Solution of(S)-2,3-bis((((9H-fluoren-9-yl) methoxy) carbonyl)amino) propanoic acid (Fmoc-Dap (Fmoc)-OH, 3.68 g, 6.71 mmol), 1-[bis(dimethylamino)methylene]-1H-1,2,3-triazolo[4,5-b]pyridinium 3-oxid hexafluorophosphate (HATU, 2.55 g, 6.71 mmol) and 2,4,6-trimethylpyridine (1.60 mL, 12.1 mmol) in DMF (40 mL) was added to resin and mixture was shaken for 2 hrs. Resin was filtered and washed with DMF (2×40 mL), dichloromethane (2×40 mL) and DMF (2×40 mL). Fmoc groups were removed by treatment with 20% piperidine in DMF (1×5 min, 1×30 min, 2×40 mL). Resin was washed with DMF (3×40 mL), 2-propanol (2×40 mL) and dichloromethane (3×40 mL). Solution of pentafluorophenyl 1-hydroxy-4-(trifluoromethyl)-1,3-dihydrobenzo[c][1,2]oxaborole-6-carboxylate (5.53 g, 13.4 mmol) and triethylamine (4.99 mL, 35.8 mmol) in DMF (40 mL) was added to resin and mixture was shaken overnight. Resin was filtered and washed with DMF (6×40 mL) and dichloromethane (10×50 mL). The product was cleaved from resin by treatment with 2,2,2-trifluoroethanol (60 mL) for 16 hrs. Resin was filtered off and washed with dichloromethane (4×50 mL). Crude product (4) was dried in vacuo and extracted with ethyl acetate (2×70 mL) and 1 M aqueous solution of potassium hydrogen sulfate (50 mL), organic phases were dried over anhydrous sodium sulfate, filtered and the solvent was evaporated. Crude product was then triturated in diethyl ether (20 mL) to give(S)-4-((S)-2,3-bis(1-hydroxy-4-(trifluoromethyl)-1,3-dihydrobenzo[c][1,2]oxaborole-6-carboxamido) propanamido)-5-(tert-butoxy)-5-oxopentanoic acid as beige solid. Yield: 1.89 g (57%). ¹H NMR spectrum (300 MHz, AcOD-d4, δH): 8.50 (s, 1H); 8.46 (s, 1H); 8.29 (s, 1H); 8.26 (s, 1H); 5.28 (d, J=2.6 Hz, 4H); 5.20 (t, J=5.9 Hz, 1H); 4.55 (dd, J=8.5 and 5.2 Hz, 1H); 4.08 (dd, J=6.0 and 2.1 Hz, 2H); 2.57-2.42 (m, 2H); 2.34-2.16 (m, 1H); 2.17-2.08 (m, 1H); 1.47 (s, 9H). LC-MS: 746.3 (M+H)+.

### Example 3: (S)-3-(2,3-Bis(1-hydroxy-4-(trifluoromethyl)-1,3-dihydrobenzo[c][1,2]oxaborole-6-carboxamido) propanamido) propanoic acid=N^{alpha}, N^{beta}-bis-(1-hydroxy-4-(trifluoromethyl)-1,3-dihydrobenzo[c][1,2]oxaborole-6-carboxamido)-L-diaminopropionante-beta-alanine

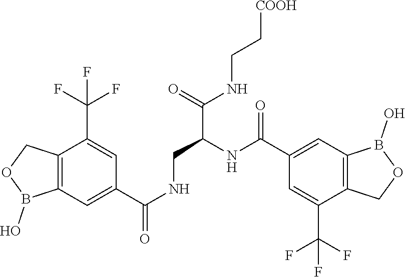 [SMILES: O=C(O)CCNC(=O)[C@H](CNC(=O)c1cc2c(c(C(F)(F)F)c1)COB2O)NC(=O)c1cc2c(c(C(F)(F)F)c1)COB2O]

A solution of L-diaminopropanoic acid hydrochloride ((S)-2,3-diaminopropanoic acid hydrochloride) (15.0 g, 107 mmol), di-tert-butyl dicarbonate (46.6 g, 214 mmol) and potassium bicarbonate (32.0 g, 320 mmol) in mixture of MeCN (400 mL) and water (400 mL) was stirred overnight. The solvent was removed under reduced pressure and the residue was acidified with saturated aqueous solution of potassium hydrogen sulfate until pH=1 was achieved. The reaction mixture was extracted with ethyl acetate (3×200 mL) and dried over anhydrous sodium sulfate. The solvent was removed under reduced pressure to give(S)-2,3-bis((tert-butoxycarbonyl)amino) propanoic acid as off-white solid. Yield: 28.2 g (87%). ¹H NMR spectrum (300 MHz, CDCl₃, δH): 5.85 (bs, 1H); 5.17 (bs, 1H); 4.31 (bs, 1H); 3.64-3.46 (m, 2H); 1.46 (s, 18H).

A solution of(S)-2,3-bis((tert-butoxycarbonyl)amino) propanoic acid (27.9 g, 91.7 mmol), tert-butyl 3-aminopropanoate (16.7 g, 91.7 mmol), N-(3-dimethylaminopropyl)-N-ethylcarbodiimide hydrochloride (EDC·HCl, 21.1 g, 110 mmol), 1-hydroxy-7-azabenzotriazole (HOAt, 15.0 g, 110 mmol) and N,N-diisopropylethylamine (64.0 mL, 367 mmol) in dichloromethane (300 mL) was stirred overnight. The solvent was removed under reduced pressure; the residue was dissolved in ethyl acetate (600 mL), washed with 1 M aqueous solution of hydrochloric acid (4×300 mL) and saturated aqueous solution of sodium bicarbonate (4×300 mL) and dried over anhydrous sodium sulfate. The solvent was removed under reduced pressure to give tert-butyl(S)-3-(2,3-bis((tert-butoxy-carbonyl)amino) propanamido) propanoate as off-white solid. Yield: 36.1 g (91%).

¹H NMR spectrum (300 MHz, CDCl₃, δH): 7.01 (bs, 1H); 5.75 (bs, 1H); 5.14 (bs, 1H); 4.15 (bs, 1H); 3.57-3.39 (m, 4H); 2.43 (t, J=6.0 Hz, 2H); 1.45 (s, 27H).

To a solution of tert-butyl(S)-3-(2,3-bis((tert-butoxycarbonyl)amino) propanamido) propanoate (36.1 g, 83.7 mmol) in dichloromethane (50 mL) was added 95% aqueous solution of trifluoroacetic acid (300 mL) and the solution was stirred for 3 hrs. The solvent was removed under reduced pressure and the residue was co-evaporated with MeCN (3×300 mL) and treated with 1 M solution of hydrogen chloride in dry diethyl ether (300 mL). The precipitate was filtered off and triturated with MeCN (2×600 mL) to give(S)-3-((2-carboxyethyl)amino)-3-oxopropane-1,2-diaminium chloride as white powder.

Yield: 22.2 g (100%). ¹H NMR spectrum (300 MHz, D₂O, δH): 4.35 (t, J=5.8 Hz, 1H); 3.63-3.46 (m, 4H); 2.67 (t, J=6.6 Hz, 2H).

To a solution of(S)-3-((2-carboxyethyl)amino)-3-oxopropane-1,2-diaminium chloride (6.41 g, 24.3 mmol) and triethylamine (33.8 mmol, 243 mmol) in water (50 mL) was added a solution of pentafluorophenyl 1-hydroxy-4-(trifluoromethyl)-1,3-dihydrobenzo[c][1,2]oxaborole-6-carboxylate (from example 2, 20.0 g, 48.6 mmol) in 1,4-dioxane (100 mL) and the solution was stirred overnight. The reaction mixture was partitioned between ethyl acetate (300 mL) and 1 M aqueous solution of potassium hydrogen sulfate (1500 mL). The organic layer was washed with 1 M aqueous solution of potassium hydrogen sulfate (1×300 mL) and the solvent was removed under reduced pressure. The residue was triturated with diethyl ether (2×150 mL) and filtered. The solid was dissolved in 70% aqueous MeCN (600 mL) and freeze-dried to give(S)-3-(2,3-bis(1-hydroxy-4-(trifluoromethyl)-1,3-dihydrobenzo[c][1,2]oxaborole-6-carboxamido) propanamido) propanoic acid as white powder. Yield: 12.1 g (80%).

¹H NMR spectrum (300 MHz, AcOD-d4, δH): 8.51 (s, 1H); 8.47 (s, 1H); 8.29 (s, 1H); 8.27 (s, 1H); 5.28 (s, 4H); 5.15 (t, J=6.1 Hz, 1H); 4.15-3.99 (m, 2H); 3.61 (t, J=6.4 Hz, 2H); 2.67 (t, J=6.3 Hz, 2H). LC-MS: 632.0 (M+H)+.

### Preparation of Insulin Derivatives

Building block of Example 2 was activated with NHS/DIC in THF and the tBu protection group was removed by treatment with TFA. The TFA was then evaporated prior to conjugation with insulin.

Building block of Example 3 was activated using NHS/DIC in MeCN before conjugation with insulin.

LCMS analysis was performed using C18 column and 0.1% TFA in water as buffer A and 0.1% TFA in MeCN as buffer B.

LCMS of boron-insulin derivatives generally show various dehydrated species as the main peaks, for example [M+nH−1×m M_{water}]ⁿ⁺ for ionization state “n” with up to “m” number of benzoxaboroles.

### Example 4

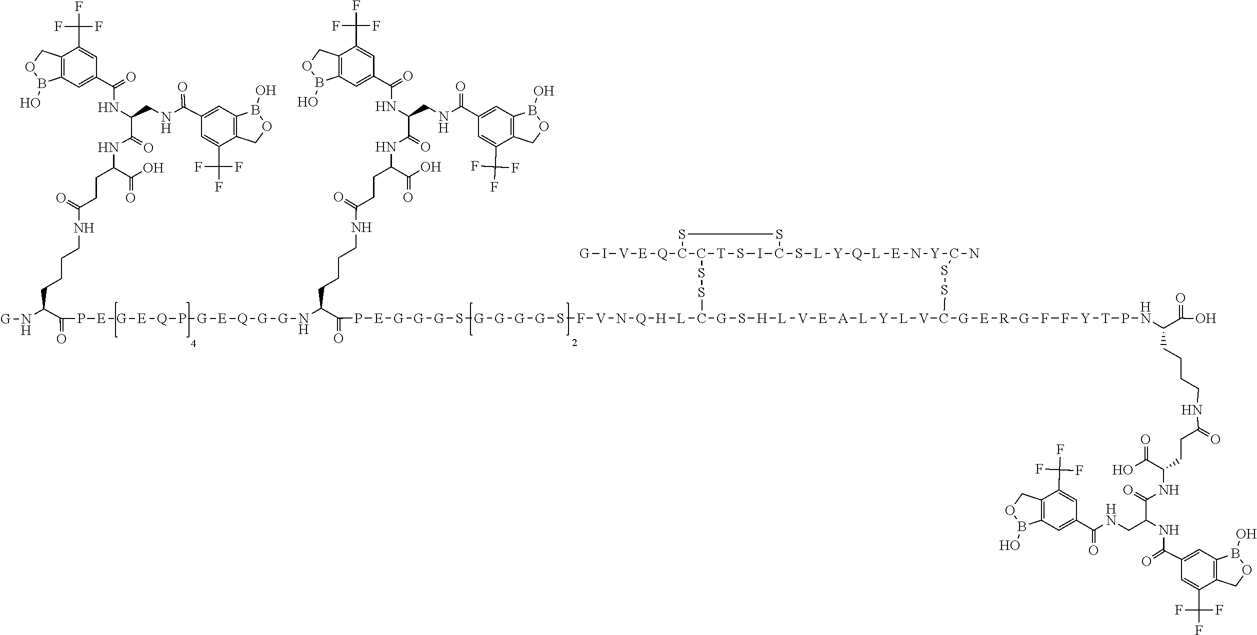

GKPE-(GEQP)₄-GEQGGKPEGGGSGGGGSGGGGS-B1 desB30 human insulin (536 mg, 0.057 mmol) was dissolved in 0.1 M Na₂HPO₄ (5.6 mL) and DMSO (2.4 mL) and pH was adjusted to 10.8 by 1.0 M aq. NaOH. Building block of example 2 (147 mg, 0.19 mmol) was NHS activated and tBu deprotected as described above, then dissolved in DMF (0.5 mL), and added dropwise over 10 mins to the given insulin solution, while pH was kept near 10.8 by dropwise addition of 0.1 M NaOH. LCMS shows formation of the desired product. The mixture was diluted with MeCN and water, and pH was adjusted to 3.8 by dropwise addition of 1 M HCl. The product was purified by reverse-phase HPLC (RP-HPLC) on C18 column using 0.1% TFA in water as buffer A and 0.1% TFA in MeCN as buffer B. The product was isolated by lyophilisation. LCMS measured 1894.1 [M+6H-5× water]⁶+, calculated 1894.1; 1623.6 [M+7H-5× water]⁷+, calculated 1623.6; 1420.8 [M+8H-5× water]⁸+, calculated 1420.8; and 1263.0 [M+9H-5× water]⁹+, calculated 1263.0, for C₄₈₀H₆₆₆B₆F₁₈N₁₂₂O₁₆₉S₆.

### Example 5 (Comparative Compound)

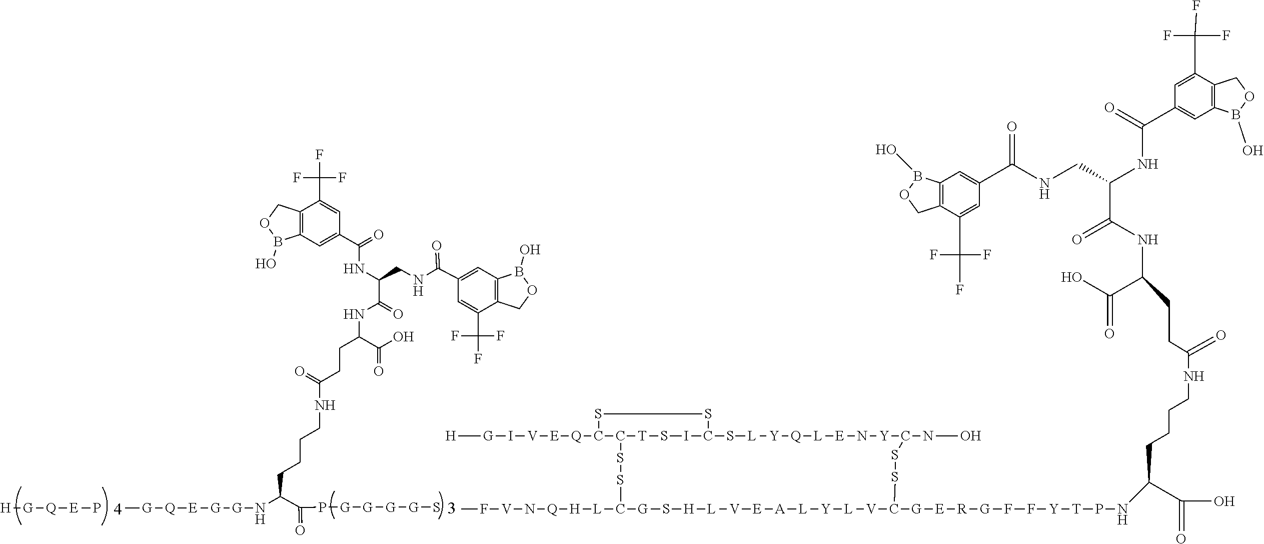

Insulin derivative of example 5 was prepared similarly to insulin derivative of example 4 from (GQEP)₄-GQEGGKPGGGGSGGGGSGGGGS-B1 desB30 human insulin and building block of example 2. LCMS of the product measured 2049.0 [M+5H-3× water]⁵+, calculated 2049.0.

### Example 6 (Comparative Compound)

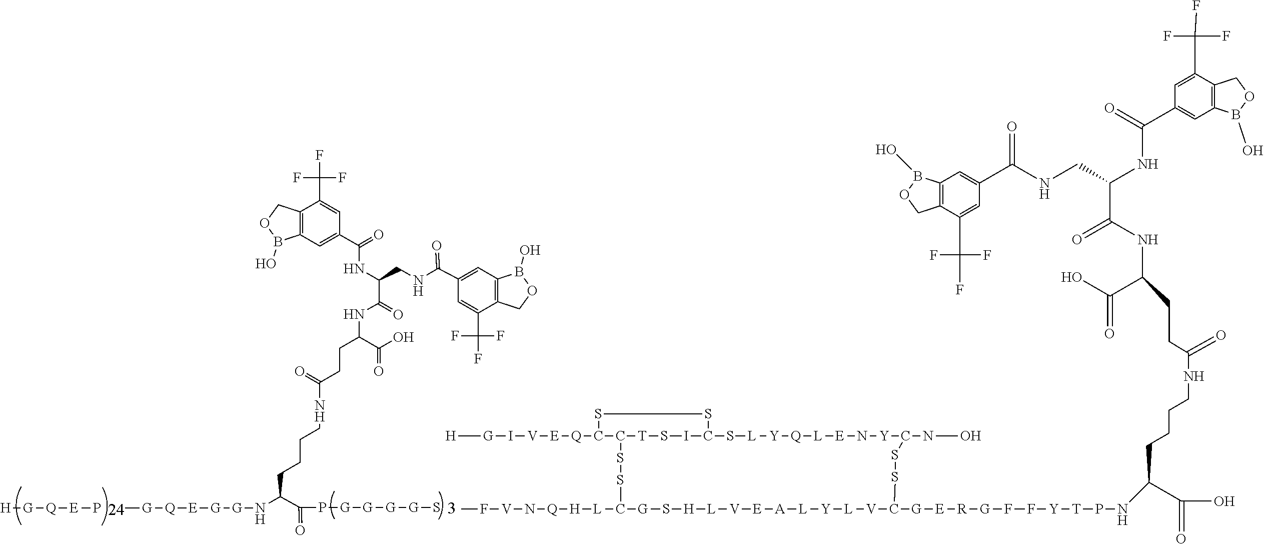

Insulin derivative of example 6 was prepared similarly to insulin derivative of example 4 from (GQEP)₂₄-GQEGGKPGGGGSGGGGSGGGGS-B1 desB30 human insulin and building block of example 2. LCMS of the product measured 1847.8 [M+10H-3× water]¹⁰+, calculated 1847.8.

### Example 7 (Comparative Compound)

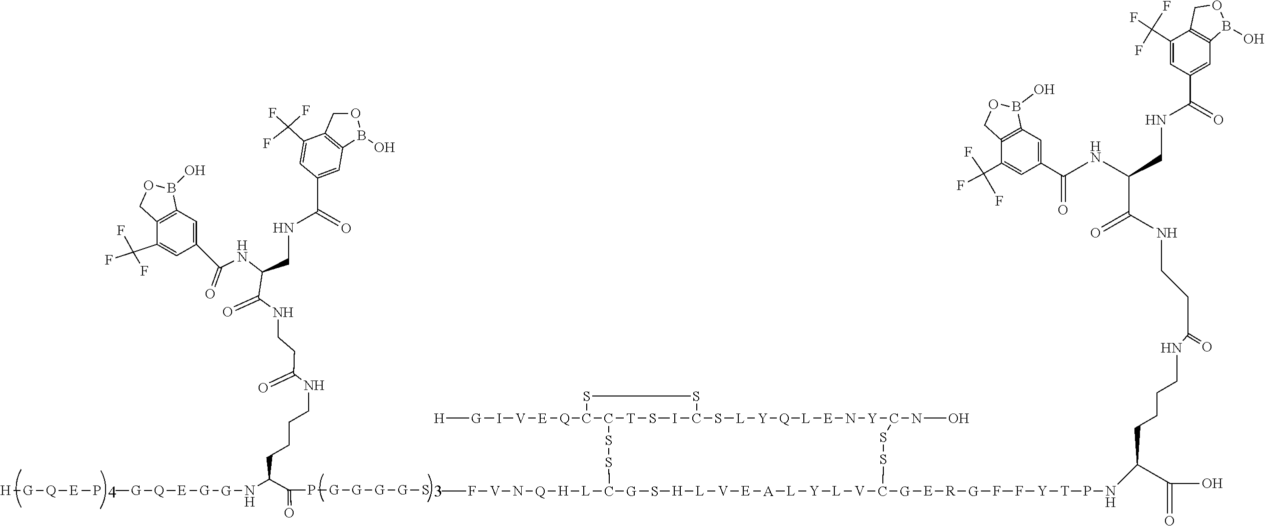

Insulin derivative of example 7 was prepared similarly to insulin derivative of example 4 from (GQEP)₄-GQEGGKPGGGGSGGGGSGGGGS-B1 desB30 human insulin and building block of example 3. LCMS of the product measured 2029.2 [M+5H-2× water]⁵+, calculated 2029.4.

### Example 8 (Comparative Compound)

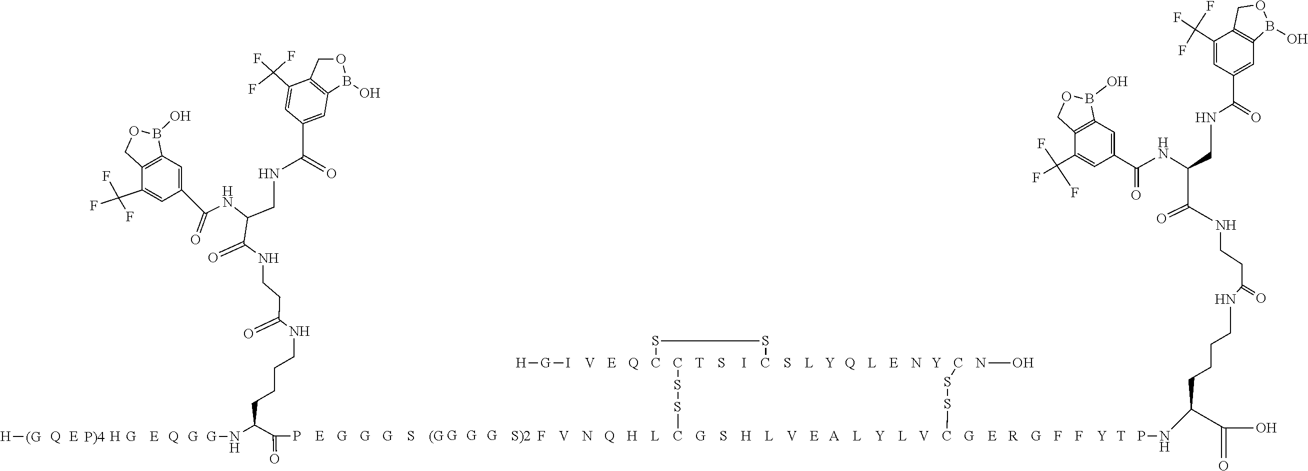

Insulin derivative of example 8 was prepared similarly to insulin derivative of example 4 from (GEQP)₄-GEQGGKPEGGGSGGGGSGGGGS-B1 desB30 human insulin and building block of example 3. LCMS of the product measured 2036.3 [M+5H-4× water]⁵+, calculated 2036.6.

### Example 9 (Prior Art Compound)

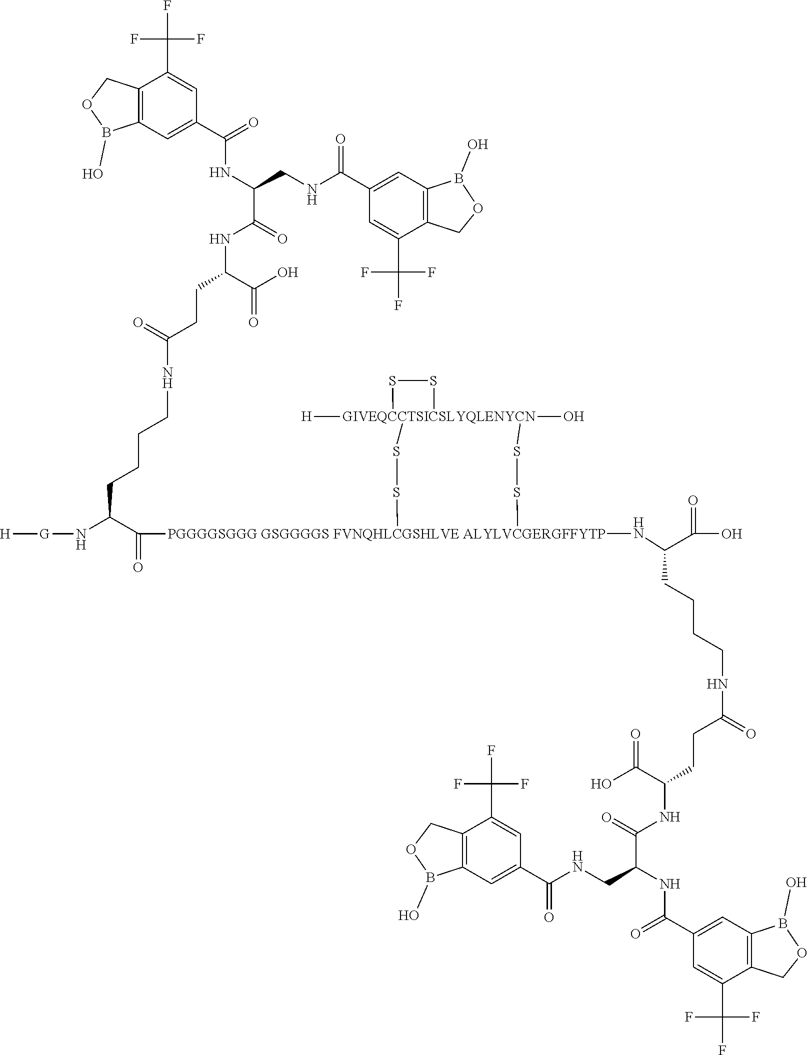

The insulin derivative shown here is the compound of Example 324 in WO2020201041. It is a prior art compound, but the structure is shown here for easy reference.

### Example 10 (Prior Art Compound)

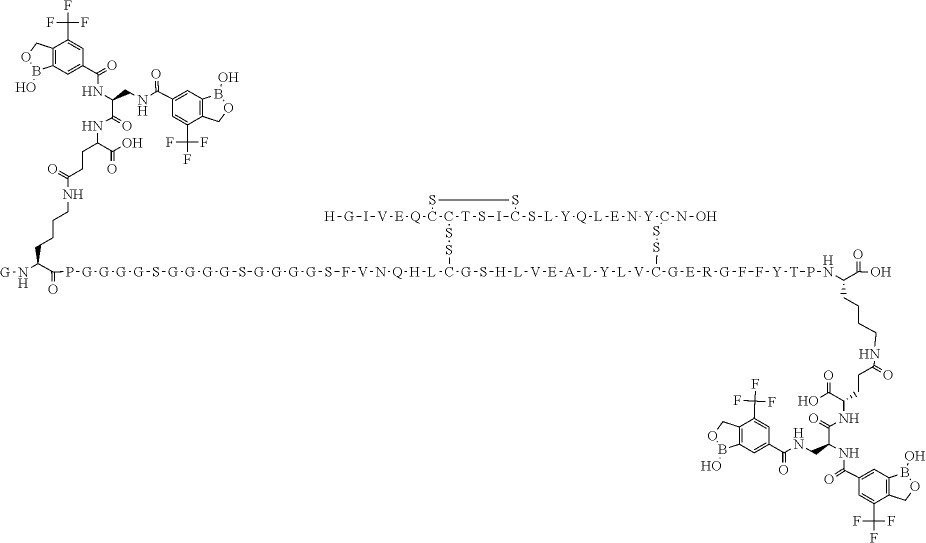

The insulin derivative shown here is the compound of Example 280 in WO2020201041. It is a prior art compound, but the structure is shown here for easy reference.

### Example 11: ALP Cleavage Rate

When insulin is produced recombinantly, it is expressed as a single-chain precursor which is later matured into 2-chain insulin using proteases, such as Achromobacter lyticus lysyl-specific endoprotease EC 3.4.21.50 (ALP) or trypsin. ALP specifically and exclusively cleaves lysyl bonds including lysyl-proline bonds (Norioka & Sakiyama (1993) Lysine-specific serine protease from Achromobacter Lyticus: Its substrate specificity and comparison with trypsin. Methods in Protein Sequence Analysis, pp. 101-106; Masaki et al. (1978) A New Proteolytic Enzyme from Achromobacter lyticus M497-I. Agricultural and Biological Chemistry, pp. 1443-1445).

The amino acid residues (AAs) in the peripheral positions, Pₙ-P₁, on the left-hand side and P₁′-Pₙ′ on the right-hand side of the cleavage site can be varied over a very large range without affecting the enzyme's ability to cleave (Sakiyama & Masaki (1994) Lysyl Endopeptidase of Achromobacter lyticus. Methods in Enzymology, Vol 244, pp. 126-137).

The cleavage rate is however affected by certain adjacent amino acids. For instance, cleavage proceeds slowly when a proline is found in position P1′ or when 1 or 2 basic amino acids precede the lysine residue (Tsusanawa et al. (1987), Amino Acid Sequence if Thermostable Direct Hemolysin Produced by Vibrio parahaemolyticus, Journal of Biochemistry, vol. 101, pp. 111-121; Sakiyama & Masaki (1994), Lysyl Endopeptidase of Achromobacter lyticus. Methods in Enzymology, Vol 244, pp. 126-137).

In this example, the effect on the ALP cleavage rate of having glutamic acid (E) in the second position (P2′) after the lysine (K) was determined. The reference insulin precursor contained a glycine (G) in P2′. In both cases the amino acid in the first position after the cleavage site (P1′) was proline (P).

To determine the ALP cleavage rate, the two insulin precursors in Table 1 were cleaved with ALP and the formation of cleavage products was followed over time by UPLC-MS (results shown in Table 2 and Table 3).

TABLE 1
Insulin precursors used for investigating the
effect of having E in position P2′ on the
ALP cleavage rate. Both precursors contain 3
cleavage sites indicated by ′ at positions A8,
AA32, and AA77.

| Insulin | | |
|---|---|---|
| precursor | Comment | Sequence |
| 1 | Insulin | EEAEPRGK'PEGEQPGEQPGEQPGEQPGEQGGK'PE |
| | precursor | GGGSGGGGSGGGGSFVNQHLCGSHLVEALYLVCGER |
| | with KPE | GFFYTPK'SDDMRGIVEQCCTSICSLYQLENYCN |
| | | (SEQ ID NO: 20) |
| 2 | Insulin | EEAEPRGK'PGGEQPGEQPGEQPGEQPGEQGGK'PG |
| | precursor | GGGSGGGGSGGGGSFVNQHLCGSHLVEALYLVCGER |
| | with KPG | GFFYTPK'SDDMRGIVEQCCTSICSLYQLENYCN |
| | (Comparative | (SEQ ID NO: 21) |
| | insulin precursor) | |

The insulin precursors were produced in Saccharomyces cerevisiae. In brief, DNA fragments encoding the relevant insulin precursors were cloned into vectors designed for guiding recombinant proteins into the secretory pathway of S. cerevisiae. The constructs were transformed into a S. cerevisiae strain which was subsequently cultivated in a defined glucose media prepared as described by Verduyn et al. (1992). Effect of Benzoic Acid on Metabolic Fluxes in Yeasts: A Continuous-Culture on the Regulation of Respiration and Alcoholic Fermentation. Yeast, vol. 8, pp. 501-517, except 6% glucose was added. Supernatants containing the insulin precursors were adjusted to pH 8.5 using 1M NaHCO₃ (pH 9.5) and diluted with water to a final concentration of 150 mg precursor/L. ALP was added to the pH adjusted supernatants to a final concentration of 0.1 mg/ml and the reaction was followed over time by taking out samples at the timepoints 0 min, 30 min, 60 min, 120 min, 240 min, 360 min and 1440 min. To stop the ALP reaction, supernatants were diluted with 1 volume 4M acetic acid. The cleavage products in the ALP cleaved samples were identified and quantified using UPLC-MS. The results are shown in Table 2 and Table 3.

The desired cleavage product is denoted ‘open insulin precursor’ and is cleaved only at the Lys residue corresponding to the Lys in position B29 of human insulin (the Lys residue at position 77 in SEQ ID NO:20 and in SEQ ID NO:21, respectively (also denoted A77)). For the open insulin precursor 1, the sequence of the A-chain is thus SDDMRGIVEQCCTSICSLYQLENYCN (SEQ ID NO:22) and the sequence of the B-chain is EEAEPRGKPEGEQPGEQPGEQPGEQPGEQGGKPEGGGSGGGGSGGGGSFVNQHLCGS HLVEALYLVCGERGFFYTPK (SEQ ID NO:23). The term ‘over-cleaved insulin precursor’ is used to denote cleavage products which have in addition to cleavage at AA77, additionally been cleaved at the Lys residue in position 8 (also denoted AA8) and/or position 32 (also denoted AA32) in SEQ ID NO:20 and in SEQ ID NO:21, respectively.

The open insulin precursor can be purified and chemically modified at the lysine residues and then fully matured by trypsin cleavage of the N-terminal extensions on the B- and A-chain, respectively (this concept is described in WO2005/047508A1). For the specific open insulin precursor 1 of this example, covalently conjugation at the three lysine residues with the appropriate modifying group, followed by cleavage of the N-terminal extensions EEAEPR and SDDMR by trypsin, would result in the insulin derivative of Example 4.

It was found that the ALP cleavage rate is greatly affected by the amino acid in position P2′. If P2′ is glycine (G), cleavage proceeded fast at all three lysine residues and over-cleavage was observed already after 60 minutes, long before AA77 was fully cleaved (Table 3). On the contrary, cleavage proceeded very slowly if the AA in position P2′ is glutamic acid (E). At timepoints 60 min and 120 min, AA77 is nearly 100% cleaved, while no cleavage at residue AA8 and AA32 could be detected (Table 2). Cleavage at AA8 and AA32 is only observed after extended incubation time for longer than 240 min and in all cases only to a limited degree.

In conclusion, precursor-loss due to ALP over-cleavage at the lysine residues AA8 and AA32 can be significantly reduced by having PE at position P1′-P2′ compared to when having PG in the same position.

TABLE 2
ALP cleavage of Precursor 1.

| Incubation time | Insulin | Open insulin | Over-cleaved insulin |
|---|---|---|---|
| with ALP | precursor 1 | precursor 1 | precursor 1 |
| (min) | (%) | (%) | (%) |
| 0 | 100 | 0.0 | 0 |
| 15 | 5.2 | 94.8 | 0.0 |
| 30 | 2.3 | 97.7 | 0.0 |
| 60 | 1.2 | 98.8 | 0.0 |
| 120 | 1.2 | 98.8 | 0.0 |
| 240 | 0.9 | 98.6 | 0.5 |
| 360 | 0.8 | 98.2 | 1.0 |
| 1440 | 0.9 | 92.7 | 6.4 |

TABLE 3
ALP cleavage of Precursor 2 (comparative insulin precursor).

| Incubation time | Insulin | Open insulin | Over-cleaved insulin |
|---|---|---|---|
| with ALP | precursor 2 | precursor 2 | precursor 2 |
| (min) | (%) | (%) | (%) |
| 0 | 100.0 | 0.0 | 0.0 |
| 15 | 40.2 | 59.8 | 0.0 |
| 30 | 31.8 | 68.2 | 0.0 |
| 60 | 16.7 | 81.3 | 1.9 |
| 120 | 3.7 | 90.7 | 5.6 |
| 240 | 0.2 | 85.5 | 14.3 |
| 360 | 0.0 | 80.6 | 19.4 |
| 1440 | 0.0 | 47.5 | 52.5 |

### Functional Data for the Insulin Derivatives

### Example 12: Assay to Determine Affinity to the Human Insulin Receptor (HIR-A) in Absence or Presence of Glucose

### Insulin Receptor Preparation

Baby hamster kidney cells (BHK) cells over-expressing human Insulin Receptor A (HIR-A) were lysed in 50 mM Hepes pH 8.0, 150 mM NaCl, 1% Triton X-100, 2 mM EDTA and 10% glycerol. The cleared cell lysate was batch absorbed with wheat germ agglutinin (WVGA)-agarose (Lectin from Triticum vulgaris-Agarose, L1394, Sigma-Aldrich Steinheim, Germany) for 90 min. The receptors were washed with 20 volumes 50 mM Hepes pH 8.0, 150 mM NaCl and 0.1% Triton X-100, where after the receptors were eluted with 50 mM Hepes pH 8.0, 150 mM NaCl, 0.1% Triton X-100, 0.5 M n-Acetyl Glucosamine and 10% glycerol. All buffers contained the protease cocktail Complete (Roche Diagnostic GmbH, Mannheim, Germany) as described in Andersen et al. 2017 PLos One 12.

### Insulin Receptor Scintillation Proximity Assay (SPA) Binding Assay

SPA PVT anti-mouse beads (Perkin Elmer) were diluted in SPA binding buffer, consisting of 100 mM Hepes, pH 7.4, 100 mM NaCl, 10 mM MgSO₄, 0.025% (v/v) Tween-20. SPA beads were incubated with the IR-specific antibody 83-7 (Soos et al. 1986 Biochem J. 235, 199-208) and solubilized semi-purified HIR-A. Receptor concentrations were adjusted to achieve 10% binding of 5000 cpm ¹²⁵I-(Tyr31)-Insulin (Novo Nordisk A/S). Dilution series of cold ligands were added to 96-well Optiplate, followed by tracer (125|-Insulin, 5000 cpm/well) and lastly receptor/SPA mix. In order to test the glucose sensitivity, the binding experiments were set up in absence or presence of 20 mM glucose. The plates were rocked gently for 22.5 hrs at 22° C., centrifuged for 5 min at 1000 rpm and counted in TopCounter (Perkin Elmer). Data points were fitted to a four-parameter logistic model, whereby the relative affinity of the insulin derivative compared to human insulin (within the same plate) was determined. The relative affinities for the insulin derivatives compared to human insulin were determined, and the increase in apparent relative affinity from 0 to 20 mM glucose reflects the glucose sensitivity of the insulin derivatives in each experiment. The experiments were done in absence and also in presence of 1.5% HSA (w/w) to better resemble physiological conditions. Data is shown in Table 4a (no HSA) and Table 4b (1.5% HSA).

The insulin derivatives of the present invention bind to HSA, and when the HIR affinity is measured in the presence of HSA, a proportion of the insulin derivative is thus bound to HSA and is not able to bind to the human insulin receptor. Thus, the term ‘apparent affinity’ is used to describe the affinity measured in the presence of 1.5% HSA.

The HIR (Human Insulin Receptor) glucose factor was determined in the individual experiment as the apparent relative HIR affinity at 20 mM glucose divided by the relative affinity in absence of glucose. The average glucose factor from several experiments is provided in Table 4a and Table 4b. The glucose factor provided in Table 4a and Table 4b is the average glucose factor determined in the individual experiments and may thus differ slightly from the value obtained by dividing the average HIR affinity in presence of glucose with the average HIR affinity in absence of glucose.

### Insulin Derivatives of the Invention

The data in Table 4a and Table 4b shows the albumin dependent glucose sensitivity. The apparent insulin receptor affinity of insulin derivative of Example 4 decreases when measured in presence of HSA compared to absence of HSA (compare data in Table 4a with data in Table 4b). Glucose can displace the binding of the insulin derivative of Example 4 to HSA, thereby increasing the apparent HIR in presence of 20 mM glucose and 1.5% HSA as compared to when no glucose is present (see data in Table 4b). This can readily be seen from the glucose factor, which is above 1 when the relative insulin receptor affinity is higher in the presence of 20 mM glucose, as compared to when no glucose is present. The insulin derivative thus binds to the insulin receptor in a glucose sensitive fashion in the presence of HSA, and the insulin derivative thus have the potential for glucose sensitive treatment of diabetes, where the insulin derivative will be inactive or less active at low blood glucose levels and will bind to and activate the insulin receptor at higher blood glucose levels. As can be seen from Table 4b, the insulin derivative of Example 4 has a high glucose factor of 51.9 in the presence of 1.5% HSA.

TABLE 4a
HIR relative affinity to Human Insulin (HI)
(%) in the absence of HSA and in the
absence and presence of glucose

| | | HIR | HIR | |
|---|---|---|---|---|
| | | affinity | affinity | |
| Insulin | Peptide extension at | relative to | relative to | |
| derivative | B1 of insulin backbone | HI (%), | HI (%), | HIR |
| of | (no of amino acid | 0 mM | 20 mM | glucose |
| Example | residues in extension) | glucose, | glucose, | factor, |
| No | (SEQ ID NO) | 0% HSA | 0% HSA | 0% HSA |
| 4 | GKPE-(GEQP)₄-GEQGGK | 62 | 45 | 0.7 |
| | PEGGGS(G₄S)₂ | | | |
| | (42 amino | | | |
| | acid residues) | | | |
| | (SEQ ID NO:4) | | | |

TABLE 4b
HIR relative affinity to Human Insulin (HI)
(%) in the presence of 1.5% HSA and in
the absence and presence of glucose

| | | HIR affinity | HIR | |
|---|---|---|---|---|
| | Peptide extension at | relative to | affinity | |
| Insulin | B1 of insulin | HI (%), | relative to | HIR |
| derivative | backbone | 0 mM | HI (%), | glucose |
| of | (no of amino acid | glucose, | 20 mM | factor, |
| Example | residues in extension) | 1.5% HSA | glucose, | 1.5% |
| No | (SEQ ID NO) | | 1.5% HSA | HSA |
| 4 | GKPE-(GEQP)₄-GEQGG | 0.1 | 6.3 | 52 |
| | KPEGGGS(G₄S)₂ | | | |
| | (42 amino | | | |
| | acid residues) | | | |
| | (SEQ ID NO:4) | | | |

### Insulin Derivatives Included for Comparison

In Table 5, data is shown for three insulin derivatives which are identical except for the peptide extension at the N-terminal of the insulin B-chain. The structures of the insulin derivatives are provided in Examples 5, 6, and 9, respectively, and the sequence of the peptide extension is shown in Table 5 for easy reference. The modifying groups of these insulin derivatives are identical to the modifying groups of the insulin derivative of Example 4. However, as compared to the insulin derivative of Example 4 of the present invention, these insulin derivatives only have 2 modifying groups. The prior art compound of Example 324 from WO2020201041 (structure shown in Example 9 for easy reference) has the peptide extension GKP (G₄S)₃ at position B1 of the insulin backbone. It can be seen that further elongating the peptide extension with 20 amino acid residues (as in the comparative insulin derivative of Example 5) results in a decrease of the glucose factor from 46.5 to 26.3. When the peptide extension is elongated even further as in the comparative insulin derivative of Example 6 with 100 amino acid residues, the glucose factor is reduced even further to 18.6. It is thus surprising that the insulin derivative of Example 4 of the present invention display a glucose factor of 51.9 even though it has a peptide extension of similar length as the insulin derivative of Example 5.

TABLE 5
Relative insulin receptor binding affinity
for comparative compounds and a prior art
compound in the absence and presence
of glucose, respectively.

| | | HIR | HIR | |
|---|---|---|---|---|
| | | affinity | affinity | |
| | Peptide extension | relative | relative | |
| | at B1 of | to | to | HIR |
| | insulin backbone | HI (%), | HI (%), | glucose |
| | (no of amino acid | 0 mM | 20 mM | factor, |
| Insulin derivative of | residues in | glucose, | glucose, | 1.5% |
| Example No | extension) | 1.5% HSA | 1.5% HSA | HSA |
| Insulin derivative of | (GQEP)₄-GQEGGKP(G₄S)₃ | 0.4 | 10.3 | 26 |
| Example 5 | (38 amino acid residues) | | | |
| (comparative compound) | (SEQ ID NO: 9) | | | |
| Insulin derivative of | (GQEP)₂₄-GQEGGKP(G₄S)₃ | 0.2 | 4.2 | 19 |
| Example 6 | (118 amino acid residues) | | | |
| (comparative compound) | (SEQ ID NO: 10) | | | |
| Insulin derivative of | GKP(G₄S)₃ | 0.3 | — | 46.5 |
| Example 9 | (18 amino acid residues) | | | |
| (prior art compound; Ex | (SEQ ID NO: 11) | | | |
| 324 from WO2020201041) | | | | |

In Table 6, data is shown for three insulin derivatives which are identical except for the peptide extension at the N-terminal of the insulin B-chain. The modifying group of these insulin derivatives are different than the modifying group of the insulin derivatives in Table 5. The structures of the insulin derivatives are provided in Examples 7, 8, and 10, respectively, and the sequence of the peptide extension is provided in Table 6 for easy reference. The prior art compound of Example 280 from WO2020201041 (structure shown in Example 10 for easy reference) has the peptide extension GKP (G₄S)₃ at position B1 of the insulin backbone.

It can be seen that further elongating the peptide extension with 20 amino acid residues in the comparative insulin derivatives of Examples 7 and 8 results in a decrease of the glucose factor from 18.0 to 12.6 and 12.0, respectively. This further supports the finding that elongating the peptide extension in prior art compounds such as Examples 324 and 280 from WO2020201041 results in a decreased glucose factor.

TABLE 6
Relative insulin receptor binding affinity for
comparative compounds and a prior art compound in the
absence and presence of glucose, respectively.

| | | HIR | HIR | |
|---|---|---|---|---|
| | Peptide extension | affinity | affinity | HIR |
| | at B1 of insulin | relative to | relative to | glucose |
| | backbone | HI (%), | HI (%), | factor, |
| | (no of amino acid | 0 mM | 20 mM | 1.5% |
| | residues in | glucose, | glucose, | HSA |
| Insulin derivative | extension) | 1.5% HSA | 1.5% HSA | |
| Insulin derivative of | (GQEP)₄-GQEGGKP(G₄S)₃ | 0.5 | 6.4 | 13 |
| Example 7 | (38 amino acid residues) | | | |
| (comparative | (SEQ ID NO: 9) | | | |
| compound) | | | | |
| Insulin derivative of | (GEQP)₄-GEQGGKPEGGGS(G₄S)₂ | 0.5 | 6.1 | 12 |
| Example 8 | (38 amino acid residues) | | | |
| (comparative | (SEQ ID NO: 12) | | | |
| compound) | | | | |
| Insulin derivative of | GKP(G₄S)₃ | 0.5 | — | 18.0 |
| Example 10 | (18 amino acid residues) | | | |
| (prior art compound; | (SEQ ID NO: 11) | | | |
| Ex 280 from | | | | |
| WO2020201041) | | | | |

### Example 13: Assay to Determine Glucose Sensitive Insulin Receptor Phosphorylation in Human Primary Hepatocytes

When insulin binds to the insulin receptor it induces activation of the insulin receptor which can be measured as tyrosine phosphorylation. This induces downstream signalling pathways leading to mitogenic and metabolic responses. The insulin derivative of the present invention binds to albumin and can be displaced by glucose. The glucose sensitivity of the insulin derivative of Example 4 can be measured at low and high glucose concentrations thereby detecting glucose dependent cellular responses of the insulin derivative of Example 4.

Donor Human cryo-plateable hepatocytes were incubated with increasing concentrations of human insulin (HI) or insulin derivative of Example 4 in presence of 1.5% human serum albumin (HSA) for 15 min. Hepatocytes were then washed, lysed and phosphorylation of insulin receptor (p-Tyr1158) were revealed using ELISA with Novo Nordisk developed specific insulin receptor antibody D2 (Ørstrup et al. 2019, Journal of Immunological Methods, Volume 465, February 2019, Pages 20-26) and IR p-Tyr1158 antibody (ThermoFischer, Cat. #44-802G).

Insulin receptor phosphorylation methods:

Human hepatocytes obtained from BioIVT (Liverpool cryo-plateable hepatocytes, ref. #X008001-P lot #ACR; single donor cryo-plateable hepatocytes #M00995P) were thawed and seeded according to manufacturer's instructions at a density of 50.000 cells/well in 0.1 mL/well of Invitrogro CP Medium supplemented with a Torpedo antibiotic mix” (Z99000). After 4 hrs, media is changed to M199 medium supplemented with 5.5 mM glucose, 100 units/ml penicillin and 100 mg/ml streptomycin, 4 mg/ml dexamethasone, 0.1% fetal calf serum (FCS) and 1 nM Human Insulin (HI) at 37° C. for overnight culture. The day after seeding, human hepatocytes were incubated in assay media corresponding to basal culture media supplemented (M199 w.o phenolred, tween 80 and adenosine-5-triphosphate) with 1.5% Human Serum Albumin (HSA), and either 3- or 20-mM D-Glucose and increasing concentrations of either HI or insulin derivative of Example 4 for 15 min at 37° C. After stimulation, hepatocytes were washed twice in ice-cold PBS, cells are then solubilized at 4° C. for 30 min by addition of lysis buffer. Subsequently, phosphorylation of insulin receptor (p-Tyr1158) was quantified using the immunoassay ELISA with IR specific antibody IR (D2 (Ørstrup et al. (2019) J. Immunological Methods 465. 20-26), batch 0268-0000-0945-1B) and detection pIR: pTyr1158 antibody (ThermoFischer, Cat. #44-802G). Absorbance at 450 nm was then read using SpectraMax_1_190, Molecular Devices.

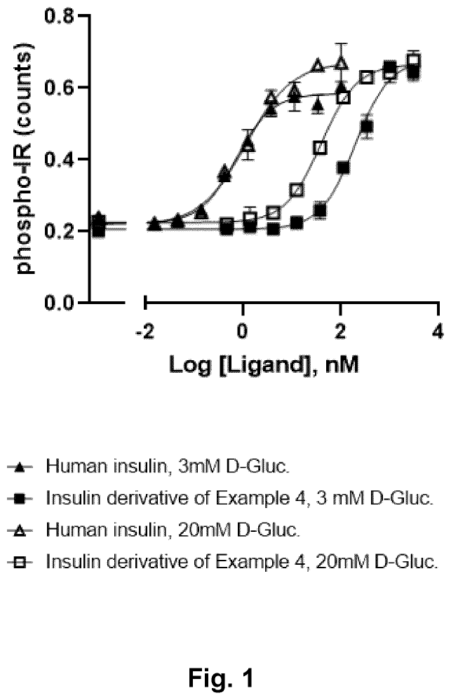

The insulin derivative of Example 4 stimulated the phosphorylation of IR (plR) in a concentration-dependent manner in human primary hepatocytes and to the same maximal level than HI albeit with a higher EC₅₀. The EC₅₀ of the insulin derivative of Example 4 was found to be lower at 20 mM (30.8 nM) than at 3 mM (58.1 nM) D-glucose concentration as revealed by a left shift of the concentration-response curve for pIR stimulation (n=6) (shown in FIG. 1). Then, the ratio between EC₅₀ at 3 mM D-glucose and the EC₅₀ at 20 mM D-glucose mM of the insulin derivative of Example 4 (IR pTyr human hepatocytes 1.5% HSA 3 vs 20 mM D-glucose factor) was calculated and was found to be 1.89 (P value=0.019) (Table 7). This demonstrates that the insulin derivative of Example 4 has a higher activity at higher levels of glucose than at lower levels of glucose, and thus that the insulin derivative of Example 4 displays glucose-sensitive insulin activity.

TABLE 7
Insulin receptor phosphorylation data

| | EC₅₀ | EC₅₀ | IR pTyr human |
|---|---|---|---|
| Insulin | 3 mM | 20 mM | hepatocytes 1.5% |
| derivative of | D-glucose | D-glucose | HSA 3 vs 20 mM |
| Example No. | (mM) | (mM) | D-glucose factor |
| 4 | 58.1 | 30.8 | 1.89 |
| | 95% Cl: | 95% Cl: | (P value = 0.019) |
| | 39.1-86.5 | 23.9-39.7 | |

### Example 14: Assay to Determine Carbohydrate-Sensitive Glucose Uptake in Cells (Rat Lipogenesis Assay)

When insulin binds to the insulin receptor it induces activation of downstream signaling pathways. One metabolic endpoint of insulin signaling is lipid metabolism, and the lipogenesis assay was used to measure an endpoint read-out because in presence of insulin, 3H-glucose uptake by the cells is stimulated and is incorporated into lipids.

The insulin derivative of Example 4 binds to albumin and can be displaced by glucose giving the glucose sensitivity. Both L- and D-glucose can displace the derivative from albumin, but L-glucose is metabolic inert and is used to change the glucose concentration in the media without affecting the metabolism. The fold change between the potency of the insulin derivative of Example 4 (relative to human insulin) at 20 mM and 3 mM L-glucose concentration was determined.

Rat Lipogenesis Assay (rFFC)

Epidydimal fat pads from Sprague Dawley rat were degraded with collagenase in Hepes Krebs Ringer Buffer at 36.5° C. for 1-1.5 hrs under vigorous shaking. The suspension was filtered through 2 layers of gauze. The phases were separated by 5 min standing at room temperature, allowing the adipocytes to collect in the upper phase. The lower phase was removed with a syringe. The adipocytes were washed twice with 20 ml Hepes Krebs Ringer Buffer. Cells were transferred to 96 well plates in Hepes Krebs Ringer buffer containing 1.5% HSA, 0.5 mM glucose, 0.1 μCi/well glucose (D-[3-³H]glucose (20.0 Ci/mmol) Perkin Elmer), and 3 mM or 20 mM L-Glucose. Increasing amounts of human insulin or insulin derivatives of the invention to generate concentration-response curves were added and incubated for 2 hrs at 36.5° C. The reactions were stopped by addition of 100 μL Microscient E (cat #6013661 Perkin Elmer). The plates rested 3 hrs before counting in Top counter. The ratio between the EC₅₀ at 3 mM L-glucose and the EC₅₀ at 20 mM L-glucose mM of the insulin derivatives (rFFC 1.5% HSA 3 vs 20 mM L-glucose factor) was determined (see Table 8).

TABLE 8
Rat lipogenesis assay data.

| Insulin derivative of | rFFC 1.5% HSA 3 vs |
|---|---|
| Example No. | 20 mM L-glucose factor |
| 4 | 4.3 (SD:0.41) |

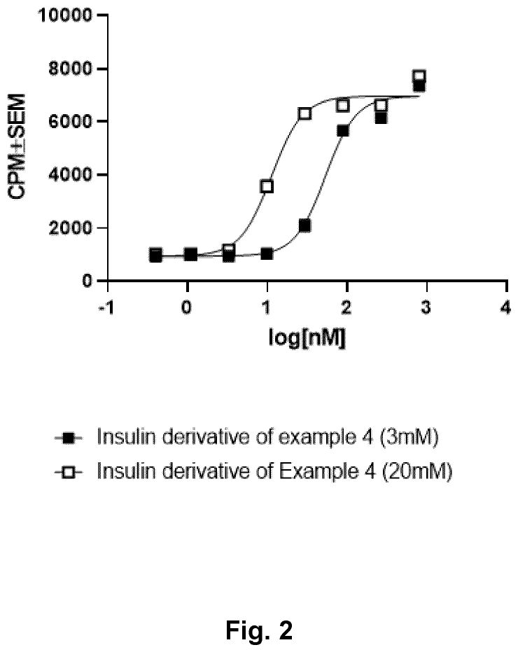

A representative concentration-response curve (n=1) is shown in FIG. 2. The EC₅₀ of the insulin derivative of Example 4 was found to be lower at 20 mM than at 3 mM L-glucose concentration as revealed by a left shift of the concentration-response curves for lipogenesis. The data in Table 8 shows that the insulin derivative of Example 4 give a higher level of lipogenesis (i.e. more glucose transport) in the presence of higher levels of L-Glucose (20 mM) compared to low L-Glucose (3 mM). This demonstrates that the insulin derivative of Example 4 has a higher activity at higher levels of glucose than at lower levels of glucose, and thus that the insulin derivative of Example 4 displays glucose-sensitive insulin activity.

### Example 15: Assay to Determine Glycogen Accumulation in Rat Primary Hepatocytes

When insulin binds to the insulin receptor it induces activation of downstream signaling pathways leading to activation of metabolic processes in insulin sensitive tissues. Among other functions, insulin induces glycogen accumulation in the liver. Therefore, estimation of the glycogen level was used as endpoint in response to increasing dose concentrations of human insulin or insulin derivative of Example 4 in primary rat hepatocytes in presence of 20 mM glucose and 0.1% human serum albumin (HSA) for 24 hrs. Quantification of cellular glycogen content was performed using a quantitative colorimetric assay based on the principle of the Periodic Acid-Schiff's reagent (PAS) assay with Absorbance reading at 550 nm.

Glycogen accumulation (PAS assay) method:

Rat hepatocytes (Lonza, RSCP01) are thawed and seeded out at a density of 50.000 cells/well in collagen-coated 96 well plates in basal M199 culture medium (5.5 mM glucose, 100 units/ml penicillin and 100 mg/ml streptomycin and 4 mg/ml decadron) supplemented with 4% fetal calf serum (FCS) and 1 nM Human Insulin (HI) at 37° C. After ~4 hrs, media was replaced with basal M199 culture medium supplemented with 0.1% FCS and 1 nM HI for overnight culture. The day after the isolation, rat hepatocytes were incubated in assay media corresponding to basal M199 media supplemented with 100 u/ml penicillin, 100 mg/ml streptomycin, 4 mg/ml dexamethasone, 0.1% Human Serum Albumin (HSA), 14.5 mM glucose and increasing concentrations of either HI or derivative of Example 4 for 24 hrs at 37° C. After stimulation, hepatocytes were washed three times in ice-cold PBS, flash frozen with liquid nitrogen and stored at −80° C. for later analysis. To measure glycogen level, plates were then thawed, and cells solubilized in 1% Triton X100. Subsequently, 100 μl of periodic acid prepared as a solution of 0.1% periodic acid in 7% acetic acid was added to the plates for a 1.5-hour period at 37° C., protected from the light, allowing oxidation of the hydroxyl groups in glycogen. 85 μl of Schiff's reagent was then added for 5 min. to react with the oxidized moieties and plates were incubated for an additional 10 min. at room temperature to allow for colour development. Absorbance at 550 nm was then read (spectraMax_1_190, Molecular Devices).

TABLE 9

| | Glycogen accumulation (PAS assay) |
|---|---|
| Insulin derivative of | at 20 mM D-Glucose |
| Example No. | (EC50, nM) |
| 4 | 20.4 |
| | (95% Cl: 16.0-26.1) |

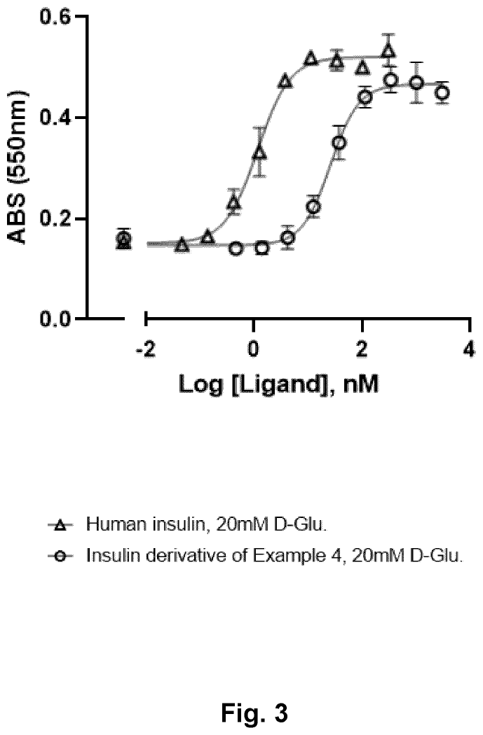

The data demonstrates that insulin derivative of Example 4 induced glycogen accumulation in rat primary hepatocytes in a concentration dependent manner and with a full dose response curve at 20 mM glucose concentration (FIG. 3). The maximal response was slightly reduced as compared to human insulin (Emax=86%, n=5). The in vitro EC₅₀ was calculated to be 20.4 nM for insulin derivative of Example 4 and 2.8 nM for human insulin (n=5) at 20 mM glucose suggesting a reduced potency relative to human insulin (Table 9).

### Example 16: Pharmacokinetic Studies in LYD Pig

### Pharmacokinetic Studies in LYD Pig

Insulin derivatives were dosed intravenously (iv) or subcutaneously (sc) to female Landrace-Yorkshire-Duroc (LYD) pigs weighed between approximately 70-110 kg. The iv dose was 0.3 nmol/kg and the sc dose either 1 or 2 nmol/kg. Blood was sampled at selected time points up to 72 hrs and plasma was prepared and analysed for insulin derivative concentration (see below). The pigs were overnight fasted and were fed 8 hrs after dosing of the insulin derivative.

Plasma concentration-time profiles from the individual animals were analysed by non-compartmental pharmacokinetics analysis (NCA) using WinNonlin Professional (Certara, CA, USA) or a custom-made Rshiny application (Rstudio.com, Boston, MA, US), validated against WinNonlin.

### Quantification of Insulin Analogue Concentrations in Plasma Samples

Plasma samples were analysed for levels of the dosed insulin derivative using Luminescence Oxygen Channeling Immunoassay (LOCI), also know under the trademark AlphaLISA (Perkin Elmer, Waltham, Massachusetts, USA). The assay principle is in short as follows: an antibody specific for the analyte of interest is conjugated to acceptor beads. A second antibody, also specific for the analyte, is biotinylated. The two antibody-conjugates are then incubated together with a plasma sample containing the analyte and an immunocomplex is formed. Next, streptavidin donor beads are added and bind to the biotinylated antibody. By illumination of the donor beads at 680 nm, ambient oxygen is excited to form singlet oxygen. If an acceptor bead is in proximity, energy transfer from singlet oxygen to the acceptor bead occurs, which in turn emits light at 615 nm. The signal strength of the emitted light is proportional to the concentration of the analyte. Plasma samples spiked with known concentration of the analyte are used to produce a calibration curve. Unknown sample concentrations are then calculated from the calibration curve, using 5 parameter logistic regression. All insulin derivatives were tested with Novo Nordisk in-house antibodies. Results are shown in Table 10.

TABLE 10
PK data

| | PK LYD pig sc |
|---|---|
| | [T½ (hrs)] |
| Insulin derivative | Mean Value |
| Insulin derivative of Example 4 | 19.0 |
| Insulin derivative of Example 9 | 13.2 |
| (prior art compound; Ex 324 from | |
| WO2020201041) | |
| Insulin derivative of Example 10 | 7.97 |
| (prior art compound; Ex 280 from | |
| WO2020201041) | |

The PK data in Table 10 shows that the insulin derivative of Example 4 of the present invention has a longer half-life than the prior art compounds of Example 324 and 280 in WO2020201041.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An insulin derivative comprising
an insulin peptide comprising B29K;
a peptide extension Z-Lys-Y-Lys-aa1-aa2-(Gly)₃-Ser-((Gly)₄-Ser)ₚ-#,
wherein Z consists of 1 to 5 amino acid residues independently selected from the group consisting of Gly, Glu, Ala, Ser, Thr, Pro, and Gln,
wherein Y consists of 15 to 30 amino acid residues independently selected from the group consisting of Gly, Glu, Ala, Ser, Thr, Pro, and Gln,
wherein aa1 is absent or Pro,
wherein aa2 is Glu or Gly,
wherein p is 1, 2, or 3, and
wherein # denotes the attachment point to the N-terminal of the B-chain of the human insulin or
human insulin analogue; and
three modifying groups of Formula A: 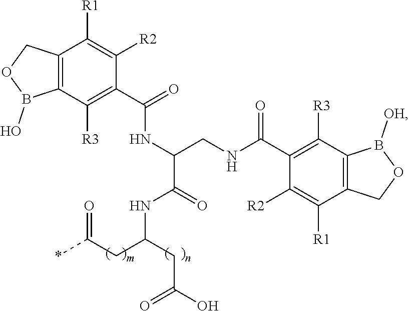
wherein one of R1, R2, or R3 is an electron withdrawing group, and the two others are hydrogen;
wherein m is 0, 1, 2, or 3;
wherein n is 0, 1, 2, or 3;
wherein * denotes the point of attachment; and
wherein one modifying group is attached to the Lys residue in position 29 of the B-chain of the insulin peptide, and the two other modifying groups are attached to each of the Lys residues in the peptide extension.

2. The insulin derivative according to claim 1, wherein Z is Gly.

3. The insulin derivative according to claim 1, wherein Y is PE(GEQP)₄GEQGG (SEQ ID NO:13).

4. The insulin derivative according to claim 1, wherein aa1 is Pro and aa2 is Glu.

5. The insulin derivative according to claim 1, wherein p is 2.

6. The insulin derivative according to claim 1, wherein the peptide extension is GKPE(GEQP)₄GEQGGKPEGGGS (G₄S)₂ (SEQ ID NO:4).

7. The insulin derivative according to claim 1, wherein the electron withdrawing group is selected from the group of CF₃, F, NO₂, CN, COX, SO₂X, and POX₂, wherein X is OR or NR₂, and R is H, alkyl or aryl.

8. The insulin derivative according to claim 1, wherein R1 is an electron withdrawing group and R2 and R3 are hydrogen.

9. The insulin derivative according to claim 1, wherein R1 is CF₃ and R2 and R3 are hydrogen.

10. (canceled)

11. An insulin derivative, wherein the insulin derivative is 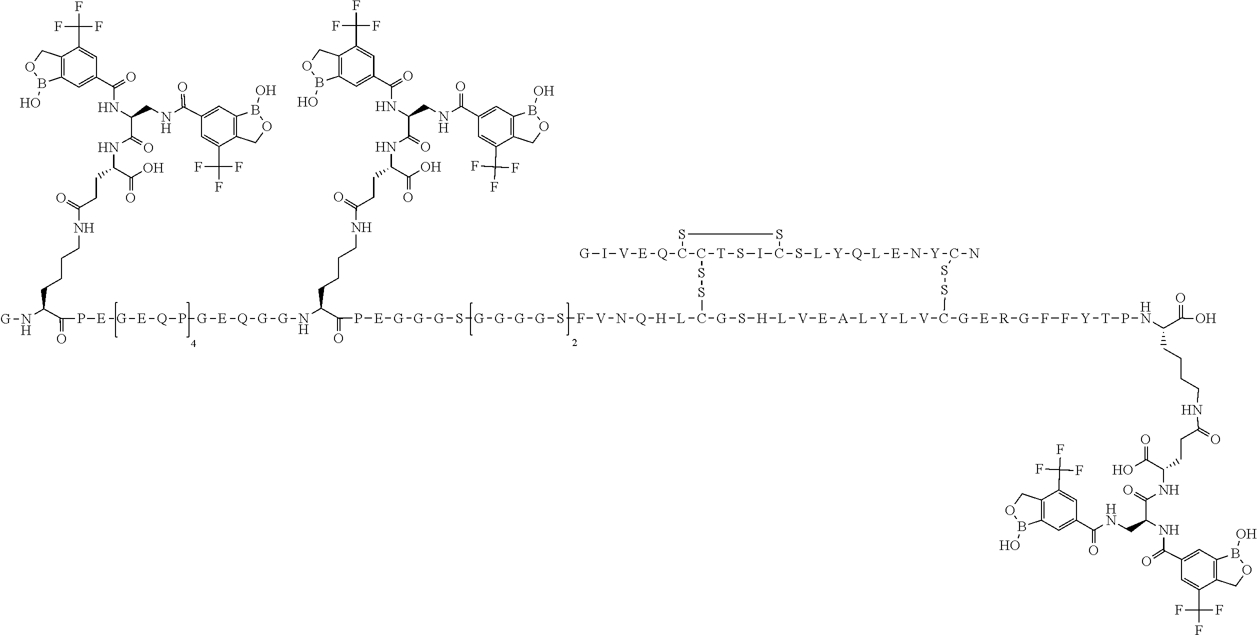
(the insulin derivative of Example 4).

12. A composition comprising an insulin derivative according to claim 1.

13. (canceled)

14. (canceled)

15. (canceled)

16. A composition comprising an insulin derivative according to claim 11.

17. A method for the treatment of diabetes, comprising administering to a subject in need thereof a therapeutically effective amount of an insulin derivative according to claim 1.

18. A method for the treatment of diabetes, comprising administering to a subject in need thereof a therapeutically effective amount of an insulin derivative according to claim 11.

19. The method of claim 17, wherein diabetes is Type 1 diabetes or Type 2 diabetes.

20. The method of claim 18, wherein diabetes is Type 1 diabetes or Type 2 diabetes.


SEQUENCE LISTING (23 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 29 aa, Homo sapiens
FVNQHLCGSHLVEALYLVCGERGFFYTPK

SEQ ID NO: 4 - protein, 42 aa, synthetic construct
GKPEGEQPGEQPGEQPGEQPGEQGGKPEGGGSGGGGSGGGGS

SEQ ID NO: 5 - protein, 71 aa, synthetic construct
GKPEGEQPGEQPGEQPGEQPGEQGGKPEGGGSGGGGSGGGGSFVNQHLCGSHLVEALYLVCGERGFFYTPK

SEQ ID NO: 6 - protein, 67 aa, synthetic construct
GQEPGQEPGQEPGQEPGQEGGKPGGGGSGGGGSGGGGSFVNQHLCGSHLVEALYLVCGERGFFYTPK

SEQ ID NO: 7 - protein, 147 aa, synthetic construct
GQEPGQEPGQEPGQEPGQEPGQEPGQEPGQEPGQEPGQEPGQEPGQEPGQEPGQEPGQEPGQEPGQEPGQEPGQEPGQEPGQEPGQEPGQEPGQEPGQEGGKPGGGGSGGGGSGGGGSFVNQHLCGSHLVEALYLVCGERGFFYTPK

SEQ ID NO: 8 - protein, 67 aa, synthetic construct
GEQPGEQPGEQPGEQPGEQGGKPEGGGSGGGGSGGGGSFVNQHLCGSHLVEALYLVCGERGFFYTPK

SEQ ID NO: 9 - protein, 38 aa, synthetic construct
GQEPGQEPGQEPGQEPGQEGGKPGGGGSGGGGSGGGGS

SEQ ID NO: 10 - protein, 118 aa, synthetic construct
GQEPGQEPGQEPGQEPGQEPGQEPGQEPGQEPGQEPGQEPGQEPGQEPGQEPGQEPGQEPGQEPGQEPGQEPGQEPGQEPGQEPGQEPGQEPGQEPGQEGGKPGGGGSGGGGSGGGGS

SEQ ID NO: 11 - protein, 18 aa, synthetic construct
GKPGGGGSGGGGSGGGGS

SEQ ID NO: 12 - protein, 38 aa, synthetic construct
GEQPGEQPGEQPGEQPGEQGGKPEGGGSGGGGSGGGGS

SEQ ID NO: 13 - protein, 23 aa, synthetic construct
PEGEQPGEQPGEQPGEQPGEQGG

SEQ ID NO: 14 - protein, 58 aa, synthetic construct
  VARIANT 5: note = G, E, A, S, T, P or Q
  VARIANT 7..21: note = G, E, A, S, T, P or Q
  VARIANT 38: note = absent or P
  VARIANT 39: note = E or G
  VARIANT 1..4: note = G, E, A, S, T, P, Q or absent
  VARIANT 22..36: note = G, E, A, S, T, P, Q or absent
XXXXXKXXXXXXXXXXXXXXXXXXXXXXXXXXXXXXKXXGGGSGGGGSGGGGSGGGGS

SEQ ID NO: 15 - protein, 53 aa, synthetic construct
  VARIANT 1..4: note = G, E, A, S, T, P, Q or absent
  VARIANT 5: note = G, E, A, S, T, P or Q
  VARIANT 7..21: note = G, E, A, S, T, P or Q
  VARIANT 22..36: note = G, E, A, S, T, P, Q or absent
  VARIANT 38: note = absent or P
  VARIANT 39: note = E or G
XXXXXKXXXXXXXXXXXXXXXXXXXXXXXXXXXXXXKXXGGGSGGGGSGGGGS

SEQ ID NO: 16 - protein, 48 aa, synthetic construct
  VARIANT 1..4: note = G, E, A, S, T, P, Q or absent
  VARIANT 5: note = G, E, A, S, T, P or Q
  VARIANT 7..21: note = G, E, A, S, T, P or Q
  VARIANT 22..36: note = G, E, A, S, T, P, Q or absent
  VARIANT 38: note = absent or P
  VARIANT 39: note = E or G
XXXXXKXXXXXXXXXXXXXXXXXXXXXXXXXXXXXXKXXGGGSGGGGS

SEQ ID NO: 17 - protein, 58 aa, synthetic construct
  VARIANT 1..4: note = G, E, A, S, T, P, Q or absent
  VARIANT 5: note = G, E, A, S, T, P or Q
  VARIANT 9..21: note = G, E, A, S, T, P or Q
  VARIANT 22..36: note = G, E, A, S, T, P, Q or absent
XXXXXKPEXXXXXXXXXXXXXXXXXXXXXXXXXXXXKPEGGGSGGGGSGGGGSGGGGS

SEQ ID NO: 18 - protein, 53 aa, synthetic construct
  VARIANT 1..4: note = G, E, A, S, T, P, Q or absent
  VARIANT 5: note = G, E, A, S, T, P or Q
  VARIANT 9..21: note = G, E, A, S, T, P or Q
  VARIANT 22..36: note = G, E, A, S, T, P, Q or absent
XXXXXKPEXXXXXXXXXXXXXXXXXXXXXXXXXXXXKPEGGGSGGGGSGGGGS

SEQ ID NO: 19 - protein, 48 aa, synthetic construct
  VARIANT 1..4: note = G, E, A, S, T, P, Q or absent
  VARIANT 5: note = G, E, A, S, T, P or Q
  VARIANT 9..21: note = G, E, A, S, T, P or Q
  VARIANT 22..36: note = G, E, A, S, T, P, Q or absent
XXXXXKPEXXXXXXXXXXXXXXXXXXXXXXXXXXXXKPEGGGSGGGGS

SEQ ID NO: 20 - protein, 103 aa, synthetic construct
EEAEPRGKPEGEQPGEQPGEQPGEQPGEQGGKPEGGGSGGGGSGGGGSFVNQHLCGSHLVEALYLVCGERGFFYTPKSDDMRGIVEQCCTSICSLYQLENYCN

SEQ ID NO: 21 - protein, 103 aa, synthetic construct
EEAEPRGKPGGEQPGEQPGEQPGEQPGEQGGKPGGGGSGGGGSGGGGSFVNQHLCGSHLVEALYLVCGERGFFYTPKSDDMRGIVEQCCTSICSLYQLENYCN

SEQ ID NO: 22 - protein, 26 aa, synthetic construct
SDDMRGIVEQCCTSICSLYQLENYCN

SEQ ID NO: 23 - protein, 77 aa, synthetic construct
EEAEPRGKPEGEQPGEQPGEQPGEQPGEQGGKPEGGGSGGGGSGGGGSFVNQHLCGSHLVEALYLVCGERGFFYTPK